(19) United States
(12) Patent Application Publication
(10) Pub. No.: US 2026/0041641 A1
(43) Pub. Date: Feb. 12, 2026

(54) NOVEL FORMULATION

(71) Applicant: GLAXOSMITHKLINE INTELLECTUAL PROPERTY DEVELOPMENT LIMITED, Stevenage (GB)

(72) Inventors: Andrew EDWARDS, Ware (GB); Sally MILLICAN, Ware (GB); Claire TRIDON, Ware (GB)

(21) Appl. No.: 19/100,150

(22) PCT Filed: Jul. 13, 2023
(86) PCT No.: PCT/EP2023/071146

§ 371 Date: Jan. 31, 2025

(30) Foreign Application Priority Data
Aug. 2, 2022 (GB) 2211231.2

(51) Int. Cl. A61K 9/20; A61K 31/515

(52) U.S. Cl. CPC A61K 9/2054; A61K 9/2013; A61K 9/2018; A61K 9/2027; A61K 9/2059; A61K 31/515

(57) ABSTRACT

The present disclosure relates to a dispersible tablet capable of disintegrating in very small volumes of water rapidly (e.g. within a period of 3 minutes) to form a suspension that is free flowing and has an acceptable mouthfeel. A dispersible tablet of daprodustat and medical uses of this tablet are also disclosed.

### FIELD OF THE INVENTION

The present disclosure relates to a dispersible tablet capable of disintegrating in very small volumes of water rapidly (e.g. within a period of 3 minutes) to form a suspension that is free flowing and has an acceptable mouthfeel. A dispersible tablet of daprodustat and medical uses of this tablet are also disclosed.

### BACKGROUND TO THE INVENTION

Whilst most adult patients are able to swallow conventional tablets and capsules, there remains a portion of patients for whom this presents difficulties. Paediatric patients, particularly those under 8 years old, frequently experience problems, as do geriatric patients and patients with certain conditions affecting swallowing (for example, neurological patients, patients with a naso-gastric tube and those with particular conditions, such as head and neck cancer).

Alternative formulation types known in the art, including suspensions, dispersible tablets and orodispersible tablets. Dispersible tablets are tablets that disintegrates in water (or potentially, other suitable vehicles like milk or juice) to form a suspension which may be drunk by the patient.

For paediatric patients, it is important that the volume of the vehicle is kept low, since it is important that the entire suspension is consumed. Given some drugs may need to be taken by babies as young as 3 months, a volume of dispersion of no more than 5 mls is desirable. This may be important for certain other patients, e.g. dialysis patients, for whom fluid intake must be restricted.

Another important consideration, particularly for the paediatric population is palatability of the medicine. Whilst this is in part dependent upon the taste of the drug in question, it is also in part dependent upon the texture of the suspension formed. This is normally referred to as mouthfeel.

Accordingly, a dispersible tablet that can rapidly be dispersed in very small volumes (1-5 ml) and generate a palatable suspension with acceptable mouthfeel is highly desirable.

Daprodustat is a HIF prolyl hydroxylase inhibitor that is in development for the treatment of anemia associated with chronic kidney disease in patients on dialysis and not on dialysis. Anemia associated with chronic kidney disease does occur rarely in children, including babies as young as 3 months. As mentioned above, dialysis patients need to restrict volume intake, so for daprodustat, a dispersible tablet capable of being rapidly dispersed in very small volumes that generate a suspension with a good mouthfeel is highly desirable.

WO 2016/120258 A1 discloses a dispersible tablet of bedaquiline fumarate. It discloses that when the dispersion occurs in a small volume of fluid (1 ml to 5 ml), the resultant mixture may be described as a soft mass.

### SUMMARY OF THE INVENTION

In a first aspect, the invention provides a dispersible tablet exhibiting a solid fraction of less than or equal to 0.9 and a surface area no greater than 615 mm², which dispersible tablet comprises an extra-granular component and at least 40% (w/w in the dispersible tablet) granules, wherein:
a. the granules are ≤140 microns in diameter with a density of 0.4-0.6 g/mL and comprise up to 60% (w/w in the granules) active pharmaceutical ingredient, between 20-60% (w/w in the granules) microcrystalline cellulose and a binder, wherein the ratio of microcrystalline cellulose to the binder is ≥10:1; and
b. the extragranular component comprises at least 20% (w/w in the dispersible tablet) silicified microcrystalline cellulose and a disintegrant.

In another aspect, the invention provides a method for manufacturing a dispersible tablet of the invention.

In a further aspect, the invention provides a dispersible tablet of daprodustat, or a pharmaceutically acceptable salt thereof, and medical uses of this tablet.

### DESCRIPTION OF DRAWINGS/FIGURES

FIG. 1 is a graph showing the impact of the solid fraction of a tablet upon disintegration time. The individual lines represent different manufacturing batches of tablets having formulations 24 or 25, where the formulations in each line differ only in solid fraction.

### DETAILED DESCRIPTION OF THE INVENTION

### Definitions

The requirement that “the granules are ≤140 microns in diameter” means that the granules have a d50≤140 microns.

The term d₅₀ has its conventional meaning and can be measured by well established techniques including sedimentation field flow fractionation, photon correlation spectroscopy, laser diffraction or disk centrifugation. The d₅₀ may be related to volume distribution of the particles and in this case the phrase “the granules are ≤140 microns in diameter” refers to the situation where at least 50% of the volume of the granules have a diameter ≤140 microns. Techniques based on either volume or weight distribution typically result in roughly the same value for the average particle size. In the context of the invention, the term “insoluble material” refers to a polymeric excipient that has a solubility in water of less than or equal to 0.1 mg/ml at 25° C.

In the context of the invention, the term “soluble filler” refers to a polyol or sugar that exhibits a solubility in water of greater than or equal to 200 mg/ml at 25° C.

In the context of the invention, the term “binder” refers to a water soluble polymer (e.g. with a solubility in water of greater than or equal to 100 mg/mL) that facilitates wet granulation Typically, the polymer has a molecular weight in the range 500 Da to 2 MDa and an apparent viscosity in the range 1 to 15,000 mPa·s when in a 2% aqueous solution at 20° C.

The term “disintegrant” is a term of art. In one embodiment, it refers to an excipient that promotes disintegration (breakdown into small fragments) of the dispersible tablet when in contact with a liquid medium.

The term “sweetener” is a term of art. In one embodiment, it refers to an excipient added to the dispersible tablet to either mask the unpleasant taste of the active pharmaceutical ingredient or to enhance the perception of a sweet taste and to improve palatability in general.

The term “lubricant” is a term of art. In one embodiment, it refers to an excipient to reduce friction between the tablet press tooling (punches and die) and the tablet.

The term “approximately” as used herein in connection with a numerical value is meant to have its usual meaning in the context of the numerical value. Where necessary the word “approximately” may be replaced by the numerical value +/−5%.

Solid fraction is a term of art. The solid fraction is calculated by dividing the tablet density (g/cm³) by the input material or compression blend true density (g/cm³). The tablet density is calculated by dividing the tablet weight (mg) by the volume of the tablet (mm³). The volume of the tablet being determined using the tablet dimensions and geometry. The input compression blend true density can be measured using a Pycnometer or calculated from all individual components true density previously measured or available from the literature.

### Statement of the Invention

Dispersible tablets of the invention are capable of completely disintegrating in small volumes of water very rapidly. In one embodiment, dispersible tablets of the invention are capable of completely disintegrating in 5 mls of water within a period of 3 minutes to form a suspension with an acceptable mouthfeel. By contrast to the dispersible tablets described in WO 2016/120258, the suspension formed is liquid and freely flowing. In order to achieve this desirable combination of properties, the inventors identified a number of critical parameters that are essential for 1) rapid disintegration and 2) acceptable mouthfeel (which includes meeting the European Pharmacopeia fineness of dispersion test criteria). This work is described in the Examples.

As explained in WO2016/12058, somewhat counterintuitively, tablets composed largely of soluble materials exhibit a long disintegration time, which they attributed to the fact that tablets may take up water and form a saturated layer preventing further diffusion of solute from the saturated stagnant layer (as per Noyes Whitney's diffusion layer theory). This phenomenon was also observed by the inventors. In WO2016/12058, it was recognised that insoluble excipients were required to act as a wick that permits water to enter the tablet formulation. The results of Human Sensory Analysis Studies presented in Example 13 demonstrates, however, that the total amount of insoluble materials present in the formulation is related to mouthfeel. It also seems possible that the large amount of insoluble materials results in the dispersion of WO2016/12058 forming a soft mass instead of a freely flowing liquid. The tablets of the present invention by contrast are feely flowing and can readily be administered by syringe or feeding tube.

In one embodiment, the dispersible tablet of the invention has a total level of insoluble materials of no greater 200 mg which would appear to be a critical level for acceptable mouthfeel (see Example 13). In the context of this invention, an insoluble material is as defined above. In a more particular embodiment, where the dispersible tablet includes as the only excipients, microcrystalline cellulose, silicified microcrystalline cellulose, binder, disintegrant, a soluble filler, lubricant and an optional sweetener, the sum of the weight of microcrystalline cellulose and silicified microcrystalline cellulose should not exceed 200 mg. It is also observed that the particle size of the insoluble materials is important for mouthfeel, and in one embodiment, the sum of the weight of microcrystalline cellulose and silicified microcrystalline cellulose should not exceed 200 mg and the median particle size (d50) of the microcrystalline cellulose and silicified microcrystalline cellulose should not exceed 125 microns.

A number of parameters in the tablet are critical to permit rapid disintegration.

As shown in Examples 1 and 2, the surface area and solid fraction of the tablet are critical parameters for ensuring disintegration within 3 minutes. A surface area of less than 615 mm² is required. In other words, the tablet must be small. This is because small tablets have a relatively high surface area to volume ratio, and because the distance that water would need to travel to penetrate the “core” of the tablet is low. In one embodiment, the surface area is from 96 to 615 mm². The solid fraction is important for similar reasons. The solid fraction of the tablet is linked to the number of pores in the extragranular component of the tablet through which water can rapidly enter (without needing to rely on the wicking action of the insoluble material). These parameters together enable rapid water penetration and breakdown of the extragranular component, liberating the granules which contain the active pharmaceutical ingredient. In one embodiment, the solid fraction is from 0.8 to 0.9.

Tablet solid fraction is related to tablet tensile strength. Tablet tensile strength is provided in certain examples as a proxy for tablet solid fraction.

The diameter of the granules and the density of the granules are important parameters for rapid disintegration for similar reason to the surface area and solid fraction of the tablet. The diameter of the granule (like the surface area of the tablet) defines the distance that water would need to travel to penetrate the “core” of the granule. Example 4 shows that granules with a median diameter ≤140 microns achieve tablet disintegration within 3 minutes. The density of the granules reflects the extent of pores and channels within the granules and also impact disintegration time by providing pores and channels to allow water to rapidly enter the granule. Example 18 shows that a granule density of 0.4-0.6 g/mL permits rapid water intake into the granules, leading to granule breakdown and liberation of the active pharmaceutical ingredient. Granule density is not significantly affected by compression to form tablets, and remains within the range stated. In one embodiment, granule density refers to the granule density before tablet formation. In one embodiment, dispersible tablets comprise granules with a density of 0.5 to 0.6 mg/mL.

It is apparent from the above that the physical structure of the tablets impacts disintegration speed. The composition of the granules and extragranular component also impacts disintegration speed, as will be discussed further below.

### Intragranular Composition

As explained above, the use of insoluble material in the granule facilitates disintegration of the tablet. The exemplified tablets in WO2016/12058 employed silicified microcrystalline cellulose intragranularly. However, the use of silicified microcrystalline cellulose intragranularly is believed to interfere with the process of wet granulation limiting the control of this process that is necessary to achieve granules of the appropriate size and density. Indeed, it is noted that in WO2016/12058, the process of granule formation was complicated involving a separate binder fraction comprised of binder and wetting agent, and an “intragranular” fraction comprised of the active ingredient, silicified microcrystalline cellulose, disintegrant and glidant. When silicified microcrystalline cellulose and glidant is omitted from the granule, wet granulation is much simpler. Accordingly, the dispersible tablets of the present invention utilise microcrystalline cellulose in the granules. In one embodiment, the granules do not contain silicified microcrystalline cellulose. In one embodiment, the granules do not contain glidant. In one embodiment, the granules do not contain silicified microcrystalline cellulose or glidant.

Example 5 shows that granules may contain between 20-60% w/w (in the granules) microcrystalline cellulose (MCC; such as AVICEL PH101 or CEOLUS KG-1000), although it will be noted that in embodiments where the total level of insoluble materials are kept below 200 mg, that either the % granules or the MCC content of the granules may need to be limited. The inventors identified that the fastest disintegration time was observed with 20-60% w/w intra-granular microcrystalline cellulose, and this was also observed to improve granulation process robustness. Tablets having between 20-30% (w/w) microcrystalline cellulose have the best mouthfeel. Accordingly, in one embodiment, the invention provides a dispersible tablet wherein the granules comprise between 20-30% (w/w in the granules) microcrystalline cellulose. In a more particular embodiment, the invention provides a dispersible tablet wherein the granules comprise approximately 20% (w/w in the granules) microcrystalline cellulose. 20% w/w MCC shows optimum granulation process robustness.

A number of different grades of microcrystalline cellulose are available. The skilled person would appreciate that the grade selected should exhibit good compressibility and mouthfeel. Mouthfeel is in part dependent upon the size of the particles. A particle size (d50) of no more than 125 microns is desirable and a size of no more than 50 microns would be ideal. AVICEL PH101 and CEOLUS KG-1000 are suitable grades. CEOLUS KG-1000 is the finest microcrystalline cellulose grade, and results in improved mouthfeel.

In addition to containing active pharmaceutical ingredient and microcrystalline cellulose, the granules must additionally comprise a binder. The binder may be a natural polymer such as a polysaccharide or polypeptide or a derivative thereof, or a synthetic polymer such as a polyalkylene oxide (e.g. PEG), polyacrylate, polyvinylpyrrolidone, or a mixture thereof. Mixed polymers, e.g. block copolymers and glycopeptides may also be used. In one embodiment, the binder may be selected from the group consisting of hydroxypropylmethylcellulose, povidone, a maltodextrin, starch 1500 or a mixture thereof. In one embodiment, the binder may be selected from the group consisting of hydroxypropylmethylcellulose, povidone, a maltodextrin or starch 1500. In another embodiment, the binder may be selected from the group consisting of hydroxypropylmethylcellulose and povidone or a mixture thereof. In another embodiment, the binder may be selected from the group consisting of hydroxypropylmethylcellulose and povidone.

In one embodiment, the binder is hydroxypropylmethylcellulose (HPMC). In one embodiment, the HPMC contains sufficient hydroxypropyl and methoxy groups to render it water-soluble. HPMC having a methoxy degree of substitution from about 19.0 to about 30.0 and a hydroxypropoxy molar substitution from about 4.0 to about 12.0 are generally water-soluble. Methoxy degree of substitution refers to the average number of methyl ether groups present per anhydroglucose unit of the cellulose molecule. Hydroxypropyl molar substitution refers to the average number of moles of propylene oxide which have reacted with each anhydroglucose unit of the cellulose molecule. A preferred HPMC is hypromellose 2910 3 mPa·s or hypromellose 2910 5 mPa·s, especially hypromellose 2910 3 mPa·s. Hydroxypropyl methylcellulose is the United States Adopted Name for hypromellose (see Martindale, The Extra Pharmacopoeia, 29th edition, page 1435). In the four digit number “2910”, the first two digits represent the approximate percentage of methoxyl groups and the third and fourth digits the approximate percentage composition of hydroxypropoxyl groups; 3 mPa·s or 5 mPa·s is a value indicative of the apparent viscosity of a 2% aqueous solution at 20° C. In one embodiment, the hydroxypropylmethylcellulose binder is hypromellose 2910 3 cP (i.e. PHARMACOAT 603).

It is believed that the use of water soluble polymeric binders may be primarily responsible for the formation of a saturated layer as discussed above. Yet, they are required in wet granulation for granule formation. The inventors have identified that the level of binders should be kept to a minimum. Example 6 shows that the ratio of microcrystalline cellulose:binder should be ≥10:1 in the granule for the tablets to disintegrate in less than 3 min, and this would appear to be most critical for tablets containing high levels of granules. In one embodiment, the invention provides a dispersible tablet wherein the granules comprise microcrystalline cellulose and a binder selected from hydroxymethylpropylcellulose or povidone in a ratio of >10:1 (the ratio is calculated based on the mass of a) the microcrystalline cellulose and b) the binder(s) present in the granules). In one embodiment, the invention provides a dispersible tablet wherein the granules comprise microcrystalline cellulose and hydroxymethylpropylcellulose in a ratio of ≥4:1. In one embodiment, the invention provides a dispersible tablet wherein the granules comprise microcrystalline cellulose and hydroxymethylpropylcellulose in a ratio of ≥10:1. In one embodiment, the invention provides a dispersible tablet wherein the granules comprise microcrystalline cellulose and binder in a ratio of between 15:1 to 10:1. In one embodiment, the invention provides a dispersible tablet wherein the granules comprise microcrystalline cellulose and binder in a ratio of approximately 10:1.

In addition, the granules may additionally contain a soluble filler and a disintegrant. In one embodiment, the invention provides a dispersible tablet, wherein the granules comprise microcrystalline cellulose, hydroxypropylmethylcellulose, one or more soluble fillers and one or more disintegrants, but no additional classes of excipients.

Suitable soluble fillers are polyols, for example, mannitol, sorbitol, maltitol, xylitol, erythritol, isomalt, lactitol and low molecular weight dextrin, and sugars such as lactose (including lactose anhydrous and lactose monohydrate), fructose, sucrose, dextrose and maltose. Accordingly, in one embodiment, the invention provides a dispersible tablet where the granules additionally comprise one or more soluble fillers selected from the group consisting of mannitol, sorbitol, maltitol, xylitol, erythritol, isomalt, lactitol, low molecular weight dextrin, lactose (including lactose anhydrous and lactose monohydrate), fructose, sucrose, dextrose and maltose. In a more particular embodiment, the invention provides a dispersible tablet where the granule additionally comprise mannitol. Mannitol is preferred over lactose as it offers better tolerability, especially in the paediatric population. However, it will be appreciated that the active pharmaceutical ingredient may limit the soluble filler selected due to incompatabilities.

The use of soluble fillers in the granule is desirable to keep the total levels of insoluble solids in the tablet low, to improve mouthfeel. Example 11 shows that 17 to 75% w/w soluble filler (e.g mannitol) have greater mechanical strength (lower level of fines) for downstream processing into tablets. Accordingly, in one embodiment, the invention provides a dispersible tablet where the granules comprise 15 to 75% (w/w in the granules) soluble filler. In a further embodiment, the invention provides a dispersible tablet where the granules comprise 17 to 75% (w/w in the granules) soluble filler. In another embodiment, the invention provides a dispersible tablet where the granules comprise 15 to 75% (w/w in the granules) mannitol. In yet another embodiment, the invention provides a dispersible tablet where the granules comprise 17 to 75% (w/w in the granules) mannitol. As will be appreciated, because widely varying levels of the soluble filler are permitted, widely differing levels of active pharmaceutical ingredient can be permitted in the granules, which can be compensated by varying the levels of the soluble filler.

Various grades of mannitol are available. Mannitol, PEARLITOL 160C produces larger and better flowing granules compared to PEARLITOL 50C or 25C.

The skilled person would appreciate that the total quantity of mannitol in the daily dose (noting that multiple tablets can be dissolved in vehicle) should be within the recommended daily allowance limit of 50 mg/kg/day (WHO Food additive series Toxicological monograph. 616. Mannitol (WHO Food Additives Series 21) (inchem.org)).

Example 12 shows that tablets comprising an intra-granular disintegrant disperse in less than 3 min. Any suitable disintegrants may be used in the granule. Suitable disintegrants include cross-linked polyvinylpyrrolidone, modified cellulose gum, e.g. croscarmellose sodium (e.g. AC-DI-SOL), sodium starch glycolate (e.g. GLYCOLYS), sodium carboxymethylcellulose, sodium dodecyl sulphate, modified corn starch, microcrystalline cellulose, magnesium aluminium silicate, alginic acid, alginate, powdered cellulose, crospovidone (such as POLYPLASDONE XL). Other disintegrants that may be considered include Xanthan gum, Gellan gum, soy polysaccharides, and the like. The optimal amount of disintegrant will depend upon which extragranular disintegrant is selected and may be readily determined by those of ordinary skill in the art. Disintegrants cause the granules to swell and promote disintegration.

In one embodiment, the disintegrant is selected from croscarmellose sodium, crospovidone XL-10 and sodium starch glycolate. In one embodiment, the disintegrant is croscarmellose sodium (such as AC-DI-SOL). In a more particular embodiment, the disintegrant is croscarmellose sodium and it is used in an amount of 1.5-3% w/w in the granule. In one embodiment, the disintegrant is crospovidone. In a more particular embodiment, the disintegrant is crospovidone and it is used in an amount of 5-15% w/w in the granule. In certain embodiments where the disintegrant is crospovidone, a fine grade crospovidone (such as POLYPLASDONE XL-10) may be used. In one embodiment, the disintegrant is sodium starch glycolate (such as GLYCOLYS). In a more particular embodiment, the disintegrant is sodium starch glycolate and it is used in an amount of 3-5% w/w in the granule. Again, it will be appreciated that the active pharmaceutical ingredient may influence the choice of disintegrant based on incompatabilities, for example, due to an incompatibility between cabotegravir and croscarmellose sodium, sodium starch glycolate was selected for use in cabotegravir granules.

### Extragranular Composition

As explained above, the use of insoluble material in the extragranular component facilitates disintegration of the tablet. The dispersible tablet of the present invention utilises silicified microcrystalline cellulose extragranularly as it has an improved mouthfeel compared to microcrystalline cellulose. Example 7 shows that tablets comprising ≥20% silicified microcrystalline cellulose (SMCC) disperse in less than 3 min. In one embodiment, the dispersible tablet contains between 20 and 50% (w/w of the dispersible tablet) silicified microcrystalline cellulose.

A number of different grades of silicified microcrystalline cellulose are available. The skilled person would appreciate that the grade selected should exhibit good flow, compressibility and mouthfeel. Mouthfeel is in part dependent upon the size of the particles. A particle size (d50) of no more than 125 microns is desirable. PROSOLV SMCC50 (d50=65 microns) and SMCC90 (d50=125 microns) are suitable grades.

In addition to containing silicified microcrystalline cellulose, the extragranular component must additionally comprise a disintegrant. The disintegrants swell when wet, and this contributes to the break up of the tablet liberating granules. Example 8 shows that different disintegrants may be employed. Suitable disintegrants include those listed above in connection with the intragranular composition, more particularly croscarmellose sodium or crospovidone XL-10. The optimal amount of disintegrant will depend upon which extragranular disintegrant is selected and may be readily determined by those of ordinary skill in the art. Example 9 shows that the disintegrant croscarmellose sodium is appropriately used in an amount of 3 to 6% w/w of the extragranular component. Accordingly, in one embodiment, the invention provides a dispersible tablet wherein the disintegrant present in the extragranular component is croscarmellose sodium (e.g. AC-DI-SOL). In a more particular embodiment, the invention provides a dispersible tablet wherein croscarmellose sodium is present in the extragranular component in an amount of 3 to 6% w/w of the tablet. 3 to 4.5% w/w croscarmellose sodium were required in the formulation for optimal dispersibility. Accordingly, in one embodiment, the invention provides a dispersible tablet wherein croscarmellose sodium is present in the extragranular component in an amount of 3 to 4.5% w/w of the tablet.

In another embodiment, the disintegrant present in the extragranular component is crospovidone XL-10. In a more particular embodiment, the disintegrant present in the extragranular component is crospovidone XL-10 and it is used in an amount of 10 to 15% w/w of the tablet. In a more particular embodiment, the disintegrant present in the extragranular component is crospovidone XL-10 and it is used in an amount of approximately 10% w/w of the tablet. As discussed above in relation to the intragranular disintegrant, the choice of extragranular disintegrant must take into account incompatibilities with the active pharmaceutical ingredient.

The extragranular component may additionally comprise a lubricant and optionally a sweetener and a soluble filler.

Suitable lubricants are pharmaceutically acceptable lubricants such as magnesium stearate, calcium stearate, stearic acid, talc, polyethylene glycol, sodium lauryl sulfate, magnesium lauryl sulphate. In one embodiment, the lubricant is sodium stearyl fumarate. In a more particular embodiment, the lubricant is sodium stearyl fumarate (e.g. PRUV) and it is used at a level of 2% (w/w) of the tablet. At this level, sodium stearyl fumarate provides efficient lubrication during tableting and also does not form a scum in the dispersion. The selection of lubricant must take into account incompatibilities with the active pharmaceutical ingredient. For example, due to incompatibility with sodium stearyl fumarate, low level of magnesium stearate (0.5% w/w) have been used successfully in daprodustat dispersible tablets. In one embodiment, the lubricant is magnesium stearate (e.g. LIGAMED). In a more particular embodiment, the lubricant is magnesium stearate and it is used at a level of 0.5% (w/w) of the tablet.

Whether a separate sweetener is required is dependent upon factors such as the taste of the active pharmaceutical agent. Taste acceptability is important for once-daily chronic dosing, particularly in the paediatric population. Accordingly, a separate sweetener may be required in the extragranular component, but it is noted that some of the possible excipients for use as soluble intragranular fillers are sugars and may also positively impact palatability. Suitable sweeteners include sugars and artificial sweeteners, for example, acesulfame potassium, neotame or sucralose. In one embodiment, the sweetener is sucralose. In a more particular embodiment, the sweetener is sucralose and is used at 1-2% w/w of the dispersible tablet formulation. In the presence of a very bitter drug, sucralose can also be used in combination with acesulfame potassium or neotame or can be replaced by neotame alone which is ˜×20 times sweeter than sucralose.

Example 7 shows that the use of a soluble filler in the extragranular component is not required, but formulation 15 shows that the use of a soluble filler can be tolerated without a negative impact upon disintegration time. Suitable soluble fillers for use in the extragranular component comprise those described above for use in the granule. In one embodiment, the soluble filler is mannitol (such as PEARLITOL 200SD). In a more particular embodiment, this is used in an amount of between 10-75% of the dispersible tablet formulation.

Coprocessed excipients (commercially available mixtures of excipients) make it more challenging to achieve the parameters required for good disintegration, and, although they could be used, are not preferred.

### Tablet

Dispersible tablets of the invention contain a minimum of 40% (w/w) granules. Example 3 shows tablets having this percentage content of granules have acceptable content uniformity. It will be appreciated by the skilled person, that content uniformity is harder to achieve for active pharmaceutical ingredients which are administered at very low dose. Since content uniformity improves with a higher granule content, the skilled person would understand that tablets containing “low dose” active pharmaceutical ingredients, may require a higher percentage of granules in order to achieve acceptable content uniformity. Indeed, as Example 3 shows, dispersible tablets having 83% (w/w) granules were shown to disintegrate within 3 minutes and exhibit good content uniformity. In one embodiment, the dispersible tablet of the invention contains between 40 and 85% (w/w) granules. In one embodiment, the dispersible tablet of the invention contains between 40 and 83% (w/w) granules. In another embodiment, the dispersible tablet of the invention contains between 40 and 60% (w/w) granules.

In one embodiment, the invention provides a dispersible tablet exhibiting a solid fraction of less than or equal to 0.9 and a surface area no greater than 615 mm², which dispersible tablet comprises an extra-granular component and at least 40% (w/w) granules, wherein:
the granules are ≤140 microns in diameter with a density of 0.4-0.6 g/mL and consist of:
i) up to 60% (w/w in the granule) active pharmaceutical ingredient;
ii) between 20-60% (w/w in the granule) microcrystalline cellulose;
iii) hydroxypropylmethylcellulose, wherein the ratio of microcrystalline cellulose to hydroxypropylmethylcellulose is ≥10:1;
iv) a disintegrant; and
v) one or more soluble fillers, and
the extragranular component consists of:
i) at least 20% (w/w in the dispersible tablet) silicified microcrystalline cellulose;
ii) a disintegrant;
iii) a lubricant;
iv) optionally a soluble filler; and
v) optionally a sweetener.

In one embodiment, the invention provides a dispersible tablet exhibiting a solid fraction of less than or equal to 0.9 and a surface area no greater than 615 mm², which dispersible tablet comprises an extra-granular component and at least 40% (w/w) granules, wherein:
the granules are ≤140 microns in diameter with a density of 0.4-0.6 g/mL and consist of:
i) up to 60% (w/w in the granule) active pharmaceutical ingredient;
ii) between 20-30% (w/w in the granule) microcrystalline cellulose
iii) hydroxypropylmethylcellulose, wherein the ratio of microcrystalline cellulose to hydroxypropylmethylcellulose is 10:1;
iv) a disintegrant selected from the group consisting of croscarmellose sodium, crospovidone XL-10 and sodium starch glycolate; and
v) a soluble filler that is mannitol, and
the extragranular component consists of:
i) at least 20% (w/w in the dispersible tablet) silicified microcrystalline cellulose;
ii) a disintegrant selected from the group consisting of croscarmellose sodium, crospovidone XL-10, sodium starch glycolate and L-HPC;
iii) a lubricant selected from the group consisting of magnesium stearate and sodium stearyl fumarate;
iv) optionally a soluble filler; and
v) optionally a sweetener.

In one embodiment, the invention provides a dispersible tablet exhibiting a solid fraction of less than or equal to 0.9 and a surface area no greater than 615 mm², which dispersible tablet comprises an extra-granular component and at least 40% (w/w) granules, wherein:
the granules are ≤140 microns in diameter with a density of 0.4-0.6 g/mL and consist of up to:
i) 60% (w/w in the granule) active pharmaceutical ingredient;
ii) between 20-30% (w/w in the granule) microcrystalline cellulose;
iii) hydroxypropylmethylcellulose, wherein the ratio of microcrystalline cellulose to hydroxypropylmethylcellulose is 10:1;
iv) a disintegrant that is croscarmellose sodium; and
v) a soluble filler that is mannitol, and
the extragranular component consists of:
i) at least 20% (w/w in the dispersible tablet) silicified microcrystalline cellulose;
ii) a disintegrant selected from the group consisting of croscarmellose sodium and crospovidone XL-10;
iii) a lubricant selected from the group consisting of magnesium stearate and sodium stearyl fumarate;
iv) optionally a soluble filler; and
v) optionally a sweetener.

In certain embodiments in which the intragranular disintegrant is croscarmellose sodium, it is used in an amount of 1.5-3% w/w of the granule. In certain embodiments in which the intragranular disintegrant is crospovidone XL-10, it is used in an amount of 5-15% w/w of the granule. In certain embodiments in which the intragranular disintegrant is sodium starch glycolate, it is used in an amount of 3-5% w/w of the granule.

In certain embodiments where the extragranular disintegrant is croscarmellose sodium, it is used in an amount from 3-6% w/w of the dispersible tablet. In certain embodiments where the extragranular disintegrant is crospovidone XL-10, it is used in an amount from 10-15% w/w of the dispersible tablet. In certain embodiments where the extragranular disintegrant is sodium starch glycolate, it is used in an amount of 5% w/w of the dispersible tablet. In certain embodiments where the extragranular disintegrant is L-HPC, it is used in an amount of 15% w/w of the dispersible tablet.

In certain embodiments where the lubricant is magnesium stearate, it is used in an amount from 0.5-1% w/w of the dispersible tablet. In certain embodiments where the lubricant is sodium stearyl fumarate, it is used in an amount of from 0.5-2% w/w of the dispersible tablet.

The tablet core may optionally be film-coated to improve taste, and/or to provide an elegant appearance. Where present, the film coat is small and in terms of weight accounts for about 3% (w/w) of the total tablet weight. It has been found that although the film coat could in theory slow water penetration into the extragranular component and into pores and channels within this, in practice, it dissolves sufficiently rapidly that it does not have an appreciable effect on disintegration time. Many suitable polymeric film coating materials are known in the art. A preferred film-coating material is hydroxypropyl methylcellulose HPMC, especially HPMC 2910 3, 5 and 6 mPa·s. Other suitable film-forming polymers also may be used herein, including hydroxypropylcellulose, and acrylate-methacrylate copolymers. Besides a film forming polymer, the film coat may further comprise a plasticizer (e.g. propylene 20 glycol) and optionally pigments (e.g. titanium dioxide, iron oxides). In one embodiment of the invention, the tablets of the invention are film coated. In another embodiment, the tablets of the invention are not film-coated.

### Suitability of the Dispersible Tablet for Different Active Pharmaceutical Ingredients

The skilled reader will readily appreciate that the dispersible tablet of the invention rapidly disintegrates, and that release of the active pharmaceutical ingredient into the vehicle results from the disintegration of the tablet that encased it. It is therefore apparent that the physical properties of the active pharmaceutical agent and in particular the solubility of the active pharmaceutical ingredient will have no impact on the disintegration time. The Examples demonstrate that three very different APIs can be formulated successfully, namely daprodustat (formulations 1, 2, 5-14, 16-20, 24 and 25), cabotegravir (formulations 3, 15, 21-23 and 26) and gepotidacin (formulation 4). These APIs have a wide range of solubility from mostly insoluble to very soluble (0.127 to 175 mg/mL). The Examples therefore show that diverse APIs can be formulated in dispersible tablets of the invention and exhibit rapid disintegration.

Despite being suitable for essentially any active pharmaceutical ingredient, it is noted that the small size of the tablets (dictated by maximum permitted surface area of the tablet) and the requirement for the granular and extragranular compositions to contain minimum amounts of particular excipients, this provides a practical limitation on the amount of active pharmaceutical composition that could be contained within a single tablet and can be calculated that dispersible tablets of the invention can accommodate a wide range of drug loading, from 0.1 to 56% w/w. Example 14 demonstrates that 4 tablets exhibit a disintegration time of less than 3 min in 5 ml water, and that increasing the volume of water to 10 ml can permit 8 tablets to be dissolved with an appropriate disintegration time. By dissolving multiple tablets, the maximum theoretical dose of active pharmaceutical ingredient can be increased (to at least 200 mg).

Accordingly, in one embodiment, the dispersible tablet of the invention comprises 0.1 to 56% w/w active pharmaceutical ingredient. In another embodiment, the dispersible tablet of the invention is suitable for active pharmaceutical compositions that have a maximum dose to be taken at a single time point of approximately 200 mg. It is also particularly suitable for active pharmaceutical ingredients typically administered to paediatric patients (particularly those under 8 years old), for geriatric patients and for patients with certain conditions affecting swallowing (for example, neurological patients, patients with a naso-gastric tube and those with particular conditions, such as head and neck cancer). Example 16 demonstrates acceptable recovery of the dose (API=daprodustat) from dosing devices used in practice, although it is noted a rinse step may be important to ensure recovery >90% from syringes and naso-gastric feeding tubes.

The skilled reader would appreciate that wet granulation could be used to form the granules used in the tablets. Compared to other formulation techniques (e.g, direct compression and dry granulation), wet granulation is complicated and usually only performed when other techniques are not suitable. Accordingly, in one embodiment, the active pharmaceutical ingredient is an active pharmaceutical ingredient that is not suitable for formulation via direct compression or dry granulation using a low shear blending process. Typically, wet granulation is particularly suitable in situations where drug loading is low, as this technique ensures that the drug substance is uniformly distributed and locked in the granules. In addition, granules produced by wet granulation are usually better flowing material than un-granulated powders (used in direct compression) or roller compacted granules. This also contributes to content uniformity because better flowing granules ensure better tablet weight control during compression. Very low dose tablets may require a high shear blending followed by high shear wet granulation to ensure the drug substance is uniformly distributed and locked in the granules.

### Daprodustat Dispersible Tablet

In one embodiment, the dispersible tablet of the invention comprises daprodustat or a pharmaceutically acceptable salt thereof. Daprodustat is the USAN, INN and JAN name for the compound N-[(1,3-dicyclohexyl-6-hydroxy-2,4-dioxo-1,2,3,4-tetrahydro-5-pyrimidinyl) carbonyl]glycine (the IUPAC name for this compound is N-[(1,3-Dicyclohexylhexahydro-2,4,6-trioxopyrimidin-5-yl) carbonyl]glycine). Daprodustat exhibits keto/enol tautomerism. All tautomers of daprodustat, including mixtures thereof, are intended to be encompassed within the scope of the invention. In a more particular embodiment, the daprodustat dispersible tablet of the invention comprises between 0.25 and 12 mg daprodustat or a pharmaceutically acceptable salt thereof (measured as the free acid). In a more particular embodiment, the daprodustat dispersible tablet of the invention comprises either 0.25 or 2 mg daprodustat of a pharmaceutically acceptable salt thereof (measured as the free acid).

Daprodustat or pharmaceutically acceptable salts thereof may be prepared in accordance with the process disclosed in WO2007/150011. In one embodiment, the tablet contains between 0.25 and 2 mg daprodustat free acid.

In a particular embodiment, the daprodustat free acid is a non-solvated crystalline form characterised by:
1) a sharp melting point from 240-242° C. as measured by thermogravimetric analysis; and/or
2) an X-ray powder diffraction (XRPD) pattern comprising at least five diffraction angles, when measured using Cu K_{α} radiation, selected from the group consisting of 4.0+/−0.2, 6.4+/−0.2, 7.5+/−0.2, 8.0+/−0.2, 15.2+/−0.2, 17.2+/−0.2, 18.6+/−0.2, 19.3+/−0.2, 19.9+/−0.2, 20.4+/−0.2, 21.0+/−0.2 and 24.1+/−0.2 degrees 2θ.
   This crystalline form may be prepared according to the process described in examples 1˜4 of WO2019052133.

In a particular embodiment, the non solvated crystalline form of daprodustat free acid is characterised by an X-ray powder diffraction (XRPD) pattern comprising at least five diffraction angles, when measured using Cu K_{α} radiation, selected from the group consisting of 4.0+/−0.2, 6.4+/−0.2, 7.5+/−0.2, 8.0+/−0.2, 15.2+/−0.2, 17.2+/−0.2, 18.6+/−0.2, 19.3+/−0.2, 19.9+/−0.2, 20.4+/−0.2, 21.0+/−0.2 and 24.1+/−0.2 degrees 2θ.

In a particular embodiment, the non solvated crystalline form of daprodustat free acid is characterised by an X-ray powder diffraction (XRPD) pattern comprising at least 6, 7, 8 or 9 diffraction angles, when measured using Cu K_{α} radiation, selected from the group consisting of 4.0+/−0.2, 6.4+/−0.2, 7.5+/−0.2, 8.0+/−0.2, 15.2+/−0.2, 17.2+/−0.2, 18.6+/−0.2, 19.3+/−0.2, 19.9+/−0.2, 20.4+/−0.2, 21.0+/−0.2 and 24.1+/−0.2 degrees 2θ.

In one embodiment, the non solvated crystalline form of daprodustat free acid is characterised by an X-ray powder diffraction (XRPD) pattern comprising at least the following diffraction angles: 6.4+/−0.2, 7.5+/−0.2 and 8.0+/−0.2 degrees 2θ.

In one embodiment, the non solvated crystalline form of daprodustat free acid is characterised by an X-ray powder diffraction (XRPD) pattern comprising at least the following diffraction angles: 6.4+/−0.2, 7.5+/−0.2, 8.0+/−0.2, 17.2+/−0.2 and 19.3+/−0.2 degrees 2θ.

In a more particular embodiment, the non solvated crystalline form of daprodustat free acid is characterised by an X-ray powder diffraction (XRPD) pattern comprising at least the following diffraction angles: 6.4+/−0.2, 7.5+/−0.2, 8.0+/−0.2, 15.2+/−0.2, 17.2+/−0.2 and 19.3+/−0.2 degrees 2θ.

In one embodiment, the non solvated crystalline form of daprodustat free acid is characterised by an X-ray powder diffraction (XRPD) pattern comprising characteristic XRPD peaks at 2theta values of 6.4°+/−0.2°, 7.5°+/−0.2°, 7.9°+/−0.2°. The X-ray powder diffraction pattern may show one or more additional characteristic peaks at 2theta values of 17.2°+/−0.2°, 21.0°+/−0.2°, 24.0°+/−0.2° or 19.3°+/−0.2°.

In another embodiment, the daprodustat free acid is a non-solvated crystalline form referred to as CS9. Form CS9 has an X-ray powder diffraction pattern that has characteristic peaks at 2theta values of 4.6°±0.2°, 6.6°±0.2°, and 21.1°±0.2° using CuKα radiation. In a more particular embodiment, the X-ray powder diffraction pattern for form CS9 has one or more additional characteristic peaks at 2theta values of 9.4°±0.2°, 20.2°±0.2°, and 24.2°±0.2° using CuKα radiation.

Form CS9 may be prepared from the free acid according to processes described in WO2019052133.

In another embodiment, the daprodustat free acid is a crystalline form referred to as Form 3. Form 3 has an X-ray powder diffraction pattern having peaks at 2-theta values of 4.5°±0.2°, 5.6°±0.2°, 9.0°±0.2° and 16.8°±0.2° using CuKα radiation. In a more particular embodiment, the X-ray powder diffraction pattern of Form 3 has one or more additional characteristic peaks at 2-theta values selected from 8.5°±0.2°, 11.2°±0.2°, 20.6°±0.2° and 24.7°±0.2° using CuKα radiation and/or a DSC endothermic peak with T onset at about 245.3° C.

In another embodiment, the daprodustat free acid is a crystalline form referred to as Form 4. Form 4 has an X-ray powder diffraction pattern having peaks at 2-theta values of 7.2°±0.2°, 11.5°±0.2°, 21.7°±0.2°, 22.9°±0.2°, 23.3°±0.2° and 25.8°±0.2° using CuKα radiation. In a more particular embodiment, the X-ray powder diffraction pattern of Form 4 has one or more additional characteristic peaks at 2-theta values selected from 6.3°±0.2°, 12.9°±0.2°, 16.5°±0.2°, 18.1°±0.2° and 19.7°±0.2° using CuKα radiation, and/or a DSC endothermic peak with T onset at about 243.9° C.

Forms 3 and 4 may be prepared as described in WO2020102302.

In another embodiment, the daprodustat free acid is a crystalline form referred to as form M. Form M has an X-ray powder diffraction pattern that has characteristic peaks at 2theta values of 4.7°±0.2°, 6.5°±0.2°, and 6.8°±0.2° using CuKα radiation. Form M may be prepared as described in WO2021031102.

In one embodiment, the invention provides a dispersible tablet exhibiting a solid fraction of less than or equal to 0.9 and a surface area no greater than 615 mm², which dispersible tablet comprises 70-83% % (w/w) granules and an extra-granular component, wherein:
a. the granules are ≤140 microns in diameter with a density of 0.4-0.6 g/mL and consist of:
   i) up to 3% (w/w in the granules) daprodustat or a pharmaceutically acceptable salt thereof (measured as the free acid);
   ii) between 20-30% (w/w in the granules) microcrystalline cellulose;
   iii) hydroxypropylmethylcellulose, wherein the ratio of microcrystalline cellulose to hydroxypropylmethylcellulose is 10:1;
   iv) a disintegrant that is croscarmellose sodium or crospovidone XL-10;
   v) a soluble filler that is mannitol; and
b. the extragranular component consists of:
   i) at least 20% (w/w in the dispersible tablet) silicified microcrystalline cellulose;
   ii) a disintegrant selected from the group consisting of croscarmellose sodium and crospovidone XL-10;
   iii) a lubricant selected from the group consisting of magnesium stearate and sodium stearyl fumarate; and
   iv) a sweetener that is sucralose.

In certain embodiments in which the intragranular disintegrant is croscarmellose sodium, it is used in an amount from 1.5-3% w/w of the granule. In certain embodiments in which the intragranular disintegrant is crospovidone XL-10, it is used in an amount from 5-15% w/w of the granule.

In certain embodiments where the extragranular disintegrant is croscarmellose sodium, it is used in an amount from 3-6% w/w of the tablet. In certain embodiments where the extragranular disintegrant is crospovidone XL-10, it is used in an amount from 10-15% w/w of the dispersible tablet.

In certain embodiments where the lubricant is magnesium stearate, it is used in an amount from 0.5-1% w/w of the dispersible tablet. In certain embodiments where the lubricant is sodium stearyl fumarate, it is used in an amount of from 0.5-2% w/w of the dispersible tablet.

In one embodiment, the dispersible tablet is film coated. In another embodiment, it is not film coated.

In another embodiment, the invention provides a dispersible tablet exhibiting a solid fraction of less than or equal to 0.9 and a surface area no greater than 615 mm², which dispersible tablet comprises 70-83% % (w/w) granules and an extra-granular component, wherein:
a. the granules are ≤140 microns in diameter with a density of 0.4-0.6 g/mL and consist of:
   i) up to 3% (w/w in the granules) daprodustat or a pharmaceutically acceptable salt thereof (measured as the free acid);
   ii) between 20-30% (w/w in the granules) microcrystalline cellulose;
   iii) hydroxypropylmethylcellulose, wherein the ratio of microcrystalline cellulose to hydroxypropylmethylcellulose is 10:1;
   iv) a disintegrant that is croscarmellose sodium;
   v) a soluble filler that is mannitol; and
b. the extragranular component consists of:
   i) at least 20% (w/w in the dispersible tablet) silicified microcrystalline cellulose;
   ii) a disintegrant that is croscarmellose sodium;
   iii) a lubricant that is magnesium stearate; and
   iv) a sweetener that is sucralose;
      wherein the dispersible tablet is optionally film coated.

In a more particular embodiment, the invention provides a dispersible tablet exhibiting a solid fraction of less than or equal to 0.9 and a surface area no greater than 615 mm², which dispersible tablet comprises 70-83% % (w/w) granules and an extra-granular component, wherein:
a) the granules are ≤140 microns in diameter with a density of 0.4-0.6 g/mL and consist of:
   i. up to 3% (w/w in the granules) daprodustat or a pharmaceutically acceptable salt thereof (measured as the free acid);
   ii. between 20-30% (w/w in the granules) microcrystalline cellulose;
   iii. hydroxypropylmethylcellulose, wherein the ratio of microcrystalline cellulose to hydroxypropylmethylcellulose is 10:1;
   iv. 1.5-3% (w/w in the granules) croscarmellose sodium;
   v. a soluble filler that is mannitol; and
b) the extragranular component consists of:
   i. at least 20% (w/w in the dispersible tablet) silicified microcrystalline cellulose;
   ii. 3-6% (w/w in the dispersible tablet) croscarmellose sodium;
   iii. a lubricant that is magnesium stearate; and
   iv. a sweetener that is sucralose;
      wherein the dispersible tablet is optionally film coated.

In a more particular embodiment, the invention provides a dispersible tablet exhibiting a solid fraction of less than or equal to 0.9 and a surface area no greater than 615 mm², which dispersible tablet comprises 70-83% % (w/w) granules and an extra-granular component, wherein:
a) the granules are ≤140 microns in diameter with a density of 0.4-0.6 g/mL and consist of:
   i. up to 3% (w/w in the granules) daprodustat or a pharmaceutically acceptable salt thereof (measured as the free acid);
   ii. 20% (w/w in the granules) microcrystalline cellulose;
   iii. hydroxypropylmethylcellulose, wherein the ratio of microcrystalline cellulose to hydroxypropylmethylcellulose is 10:1;
   iv. 1.5-3% (w/w in the granules) croscarmellose sodium;
   v. a soluble filler that is mannitol; and
b) the extragranular component consists of:
   i. at least 20% (w/w in the dispersible tablet) silicified microcrystalline cellulose;
   ii. 3-6% (w/w in the dispersible tablet) croscarmellose sodium;
   iii. a lubricant that is magnesium stearate; and
   iv. a sweetener that is sucralose;
      wherein the dispersible tablet is film coated.

In a particular embodiment, the dispersible tablet is the tablet is formulation 1 or formulation 2, as described in the Examples that comprises daprodustat as the API. As shown in Example 14, 4 tablets may be dissolved in 5 ml and 8 tablets may be dissolved in 10 ml. This ability to dissolve multiple tablets in a small volume, resulting in a free flowing solution with an acceptable mouthfeel permits all the dose levels in the dosing regimen to be achieved. As shown in Example 15, the dispersible tablet of daprodustat is stable after when stored under conditions of 40° C./75% RH for 3 months when packed in a 60 cc HDPE bottle with 2 g desiccant. Examples 16 and 17 show that the Daprodustat dispersible tablets are stable for 2 hours after dispersion in water with good recovery achieved from various dosing devices (e.g. dosing cup, syringe and feeding tubes).

### Medical Use of Daprodustat Dispersible Tablet

Dispersible tablets of daprodustat may be used in therapy, more particularly in the treatment of anemia. In a particular embodiment, the daprodustat dispersible tablet of the invention may be used in the treatment of anemia associated with chronic kidney disease (also known as renal anemia).

Accordingly, in one embodiment, the invention provides the daprodustat dispersible tablet of the invention for use in therapy.

In another embodiment, the invention provides the daprodustat dispersible tablet of the invention for use in a method of treating anemia due to chronic kidney disease.

In yet another embodiment, the invention provides use of daprodustat or a pharmaceutically acceptable salt thereof in the manufacture of the daprodustat dispersible tablet of the invention for use in the treatment of anemia due to chronic kidney disease.

In another embodiment, the invention provides a method for the treatment of anemia due to chronic kidney disease in a subject in need thereof, comprising administering to said subject the daprodustat dispersible tablet of the invention.

In a particular embodiment, the subject is human. In one embodiment, the human is a paediatric patient, under 18 years of age. In a more particular embodiment, the human is a paediatric patient, under 12 years of age. In one embodiment, the human is a paediatric patient aged between 3 months and 12 years. In another embodiment, the human is a paediatric patient under 8 years of age. In a more particular embodiment, the human is a paediatric patient aged between 3 months and 8 years.

In one embodiment, the subject having anemia due to chronic kidney disease may be receiving dialysis, for example haemodialysis or peritoneal dialysis. In an alternative embodiment, the subject is not on dialysis. In particular embodiments, the subject may be iron deficient (TSAT≤20% and/or serum ferritin≤100 ng/ml) and additionally receiving supplemental iron therapy.

In a further embodiment, the invention provides a dosage regimen for the treatment of anemia due to chronic kidney disease which aims to maintain haemoglobin in the range 10 to 12 g/dl and provide a safe increase in haemoglobin levels where haemoglobin levels are below this. The dose is modified based on the concentration of haemoglobin determined at clinical visits using an age specific algorithm. Haemoglobin concentration may be measured by known methods for example, full blood count or HemoCue.

In one embodiment, the invention provides a dosage regimen for the treatment of anemia due to chronic kidney disease for patients aged between 3 months to 2 years of age, wherein the dispersible tablet of the invention is administered at once daily equivalent dose of either 0.125, 0.25, 0.5, 0.75, 1, 1.5, 2, 3, 4, 6 or 8 mg (dose of free acid), and wherein the dose is increased or decreased by one dose step based on the haemoglobin concentration of the patient to maintain the haemoglobin concentration of the patient within the range 10-12 g/dL. Note, that the 0.125 mg once daily equivalent dose achieved by dosing 0.25 mg three times a week (TIW). All other dose steps involve once daily dosing frequency.

In one embodiment, the invention provides a dosage regimen for the treatment of anemia due to chronic kidney disease for patients aged between 2 to 6 years of age, wherein the dispersible tablet of the invention is administered at once daily dose of either 0.25, 0.5, 0.75, 1, 1.5, 2, 3, 4, 6, 8 or potentially 12 mg (dose of free acid), and wherein the dose is increased or decreased by one dose step based on the haemoglobin concentration of the patient to maintain the haemoglobin concentration of the patient within the range 10-12 g/dL.

In one embodiment, the invention provides a dosage regimen for the treatment of anemia due to chronic kidney disease for patients aged between 6 to 12 years of age, wherein the dispersible tablet of the invention is administered at once daily dose of either 0.5, 0.75, 1, 1.5, 2, 3, 4, 6, 8 and potentially 12 or 16 mg ((dose of free acid)), and wherein the dose is increased or decreased by one dose step based on the haemoglobin concentration of the patient to maintain the haemoglobin concentration of the patient within the range 10-12 g/dL.

In particular embodiments, the haemoglobin concentration of the patient is monitored at least once every three months. In a more particular embodiment, the haemoglobin concentration of the patient is monitored at least once every 8 weeks. In more particular embodiments, the haemoglobin concentration of the patient is monitored monthly or every four weeks. The skilled person will appreciate that monitoring may be more frequent when treatment is initiated, with the frequency of monitoring decreasing once the haemoglobin concentration of the patient has stabilised within the target range (10 to 12 g/dL).

In embodiments when there is a rapid increase in the haemoglobin concentration of the patient (e.g. exceeding 2.0 g/dL within 4 weeks or an initial rise on starting exceeding 1 g/dl within 2 weeks), the dose is reduced by one dose step or interrupted.

In embodiments where the haemoglobin concentration of the patient exceeds the top end of the target range, the dose is interrupted until the haemoglobin concentration is in target range, and treatment is re-started at one dose level lower.

Clinical judgement is also important in dose increases and reductions. In embodiments where the patient is above the target range, the dose is reduced by one dose step or interrupted. In embodiments where the patient is exhibiting symptoms of anemia despite being in the range 10 to 12 g/dL, the dose can be increased by one dose step, but should not exceed 12 g/dL.

A dosage regimen for treatment of anemia due to chronic kidney disease to maintain haemoglobin concentration in the range 10-12 g/dL is provided, wherein the daprodustat dispersible tablet of the invention is administered at a once daily equivalent dose of: 0.125, 0.25, 0.5, 0.75, 1, 1.5, 2, 3, 4, 6, 8 and potentially 12 or 16 mg (dose of free acid), and wherein:
a) where the haemoglobin concentration <10 g/dl and increased <0.5 g/dl compared to the previous assessment, increase the dose by one dose step;
b) where haemoglobin is within the target range 10-12 g/dL, but the patient is still symptomatic due to their anemia, increase the dose by one dose step;
c) where the haemoglobin concentration is in the range >12 to ≤12.5 g/dL, decrease the dose by one dose step;
d) where there has been an increase in haemoglobin concentration of >2 g/dl over 4 weeks, or an increase in haemoglobin concentration of >1 g/dL over 2 weeks, the dose is reduced by one dose step;
e) where the haemoglobin concentration is stable within the target range 10-12 g/dL, maintain the same dose; and
f) where the haemoglobin concentration is >12.5 g/dL, interrupt dosing until the haemoglobin concentration is ≤12.0 g/dL and restart therapy at the next lower dose.

It will be apparent that dose adjustments will result in the daprodustat dose being increased or decreased by one dose step at a time. Those receiving the highest (maximum) dose of daprodustat who require a dose increase will maintain the same dose, while those receiving the lowest dose of daprodustat that require a dose decrease will finish daprodustat therapy.

For the avoidance of doubt, it is noted that any particular dose can be administered in a single tablet or multiple tablets. For example, the dose of 4 mg could be administered as a two 2 mg tablets. For formulations 24 (0.25 mg daprodustat dispersible tablet) and 25 (2 mg daprodustat dispersible tablet) described in the examples, it is noted that up to 4 tablets can be suspended in a 5 mL volume. Dispersal of 8 tablets require a total volume of 10 mL.

For ESA naïve patients, suitable starting doses are set out in Table 1.

TABLE 1

| Baseline | Daprodusat starting dose per age group | | |
|---|---|---|---|
| Hgb | (mg, QD) | | |
| (g/dL) | 6 to <12 years | 2 to <6 years | 3 months to <2 years |
| <9 | 2 | 1 | 0.5 |
| ≥9 | 1 | 0.5 | 0.25 |

For patients not on dialysis that are switching from ESA to daprodustat, suitable starting doses are set out in Table 2.

TABLE 2

| Prior ESA dose at Baseline | | | | | |
|---|---|---|---|---|---|
| Epoetins | | | Daprodusat starting | | |
| (incl | | Methoxy | dose per age group | | |
| biosimilars) | Darbepoetin | PEG- | (mg, QD) | | |
| (convert | alfa | epoetin beta | 6 | 2 | 3 months |
| SC to IV | (μg/4 wk | (μg/month | to <12 | to <6 | to <2 |
| U/week) | SC/IV) | SC/IV) | years | years | years |
| ≤2000 ^{e} | ≤30 | ≤40 | 0.5 | 0.25 | 0.125 |
| >2000 | >30 to 300 | >40 to 360 | 1 | 0.5 | 0.25 |
| to <20,000 | | | | | |
| ≥20,000 | >300 | >360 | 2 | 1 | 0.5 |

For patients on dialysis that are switching from ESA to daprodustat, suitable starting doses are set out in Table 3.

TABLE 3

| Prior ESA dose at Baseline | | | | | |
|---|---|---|---|---|---|
| Epoetins | | | Daprodustat starting | | |
| (including | | Methoxy | dose per age group | | |
| biosimilars) | Darbepoetin | PEG- | (mg, QD) | | |
| (convert | alfa | epoetin beta | 6 | 2 | 3 months |
| SC to IV | (μg/4 wk | (μg/month | to <12 | to <6 | to <2 |
| U/week) | SC/IV) | SC/IV) | years | years | years |
| ≤2000^{e} | ≤30 | ≤40 | 2 | 1 | 0.5 |
| >2000 | >30 to 150 | >40 to 180 | 3 | 1.5 | 0.75 |
| to <10,000 | | | | | |
| ≥10,000 | >150 to 300 | >180 to 360 | 4 | 2 | 1 |
| to <20,000 | | | | | |
| ≥20,000 | >300 | >360 | 6 | 3 | 1.5 |

### Manufacture

One aspect of the invention is a process for the preparation of dispersible tablet of the invention, which comprises: (a) preparing granules using the intragranular components mentioned herein; (c) obtaining an extra-granular fraction using the extra-granular fraction components mentioned herein, and using those fractions to prepare a tablet of the invention. In one embodiment, the granules formed in (a) do not contain silicified microcrystalline cellulose.

The granules may be prepared by any suitable method, such as direct compression, dry granulation or wet granulation. In one embodiment, a wet granulation process is employed. In wet granulation, the granules may be prepared by contacting or mixing the relevant ingredients with a vehicle which may be aqueous or non-aqueous, or a combination. In one embodiment, the vehicle is water (qs), more particularly purified water (qs). Such wet granulation process is in one embodiment, a batch high shear granulation process. Use of a batch high shear wet granulation process results in better control of granule size and a reduced levels of fines compared to top spray granulation.

As shown in Example 11, the use of a soluble filler in the granule improves wet granulation and granules comprising 17 to 75% w/w soluble filler (e.g mannitol) have greater mechanical strength (lower level of fines) for downstream processing into tablets.

Use of a batch high shear wet granulation process results in improved granule properties for dispersible tablet (flow properties, control of particle size distribution, level of fines) The obtained granulate may then be dried and sized (or sieved) after which it is mixed or blended with the components of the extragranular component (as defined herein). Such blending also inherently involves lubrication where the extra-granular layer also includes a lubricant. The blend may then be converted into tablets using a conventional tablet press. The tooling used for the tablet press will determine the surface area of the tablets.

### NUMBERED EMBODIMENTS

The dispersible table may be described by the following set of numbered embodiments:
Embodiment 1. A dispersible tablet exhibiting a solid fraction of less than or equal to 0.9 and a surface area no greater than 615 mm², which dispersible tablet comprises at least 40% (w/w) granules and an extra-granular component, wherein:
   a. the granules are ≤140 microns in diameter with a density of 0.4-0.6 g/mL and comprise up to 60% (w/w) active pharmaceutical ingredient, between 20-60% (w/w) microcrystalline cellulose and a binder, wherein the ratio of microcrystalline cellulose to the binder is ≥10:1; and
   b. the extragranular component comprises at least 20% silicified microcrystalline cellulose and a disintegrant.
Embodiment 2. A dispersible tablet according to embodiment 1, wherein the granules comprise between 20-30% (w/w) microcrystalline cellulose.
Embodiment 3. A dispersible tablet according to embodiment 2, wherein the granules comprise approximately 20% (w/w) microcrystalline cellulose.
Embodiment 4. A dispersible tablet according to any preceding embodiment, wherein the binder is selected from hydroxypropylmethylcellulose or povidone.
Embodiment 5. A dispersible tablet according to any preceding embodiment, wherein the disintegrant in the extragranular component is croscarmellose sodium or crospovidone XL-10.
Embodiment 6. A dispersible tablet according to embodiment 5, wherein croscarmellose sodium is used in an amount of approximately 3-6% w/w in the extra-granular component.
Embodiment 7. A dispersible tablet according to any preceding embodiment, wherein the granules additionally comprise one or more soluble fillers that exhibit a solubility of at least 200 mg/ml at 25° C.
Embodiment 8. A dispersible tablet according to embodiment 7, wherein the granules additionally comprise one soluble filler that is mannitol.
Embodiment 9. A dispersible tablet according to any preceding embodiment, wherein the granules additionally comprise a disintegrant.
Embodiment 10. A dispersible tablet according to any preceding embodiment, wherein the extragranular component additionally comprises a sweetener and/or a lubricant.
Embodiment 11. A dispersible tablet according to any preceding embodiment, wherein one or more tablets capable of complete disintegration in 5 ml water within a time period of 3 minutes.
Embodiment 12. A dispersible tablet according to embodiment 11, wherein the suspension prepared following complete disintegration contains less than 200 mg insoluble solid particles not greater than 125 microns per unit dose dispersed.
Embodiment 13. A dispersible tablet according to any preceding embodiment, wherein the active pharmaceutical ingredient is daprodustat or a pharmaceutically acceptable salt thereof.
Embodiment 14. A dispersible tablet exhibiting a solid fraction of less than or equal to 0.9 and a surface area no greater than 615 mm², which dispersible tablet comprises 70-83% % (w/w) granules and an extra-granular component, wherein:
   b. the granules are ≤140 microns in diameter with a density of 0.4-0.6 g/mL and consist of up to 3% w/w daprodustat or a pharmaceutically acceptable salt thereof (measured as the free acid), between 20-30% (w/w) microcrystalline cellulose, hydroxypropylmethylcellulose, a disintegrant that is croscarmellose sodium or crospovidone XL-10, and a soluble filler that is mannitol, wherein the ratio of microcrystalline cellulose to hydroxypropylmethylcellulose is 10:1; and
   c. the extragranular component consists at least 20% silicified microcrystalline cellulose, a disintegrant selected from the group consisting of croscarmellose sodium and crospovidone XL-10, a lubricant selected from the group consisting of magnesium stearate and sodium stearyl fumarate, and a sweetener that is sucralose.

### EXAMPLES

### Protocols for Measurement of Disintegration Time:

Method 1: disintegration is measured using USP method <701>.
Method 2: disintegration is measured using the patient instruction for use method where 1 to 4 tablets are dispersed in 5 ml of water and where 5 to 8 tablets are dispersed in 10 ml of water using gentle swirling for 1-3 min in a 30 mL dosing cup.

Inventors observe that disintegration times measured using methods 1 and 2 show good correlation. In Examples 1, 2, and 4 to 16, the disintegration time quoted was measured with either Method 1 or Method 2.

### Example 1

Table 4 shows that tablets with a total surface area of less than 615 mm² disperse in less than 3 min at a mean tensile strength of 2 MPa (+0.5 MPa).

TABLE 4

| | Tablet Surface Area | Tablet Disintegration Time |
|---|---|---|
| Formulation | (mm²) | (seconds) |
| 1 | 96 | 21 |
| 2 | 105 | 7 |
| 3 | 145 | 21 |
| 4 | 510 | 124 |
| 4 | 615 | 116 |

The composition of formulations 1-4 are given in Table 5. The formulations demonstrate that tablets having a tablet surface area of less than 615 mm³ are capable of rapid disintegration.

TABLE 5

| | Formu- | Formu- | Formu- | Formu- |
|---|---|---|---|---|
| | lation 1 | lation 2 | lation 3 | lation 4 |
| Component | (% w/w) | (% w/w) | (% w/w) | (% w/w) |
| Intra-granular | | | | |
| Drug substance | 0.3 | 2.7 | 7.9 | 57.8 |
| Mannitol | 74.7 | 72.3 | 55.1 | 17.2 |
| MCC | 20.0 | 20.0 | 30.0 | 20.0 |
| Hypromellose | 2.0 | 2.0 | 3.0 | 2.0 |
| Croscarmellose | 3.0 | 3.0 | 0 | 3.0 |
| sodium | | | | |
| Sodium starch | 0 | 0 | 4.0 | 0 |
| glycolate | | | | |
| Total | 100.0 | 100.0 | 100.0 | 100.0 |
| Granule | 75.0 | 75.0 | 44.4 | 56.0 |
| Extra-granular | | | | |
| SMCC/MCC | 20.5 | 20.5 | 42.6 | 33.0 |
| Croscarmellose | 3.0 | 3.0 | 0 | 6.0 |
| sodium | | | | |
| Crospovidone XL-10 | 0 | 0 | 10.0 | 0 |
| Sucralose | 1.0 | 1.0 | 1.0 | 1.5 |
| Acesulfame | 0 | 0 | 0 | 3.0 |
| potassium | | | | |
| Magnesium Stearate | 0.5 | 0.5 | 0 | 0 |
| Sodium stearyl | 0 | 0 | 2.0 | 0.5 |
| fumarate | | | | |
| Total | 100.0 | 100.0 | 100.0 | 100.0 |

### Example 2

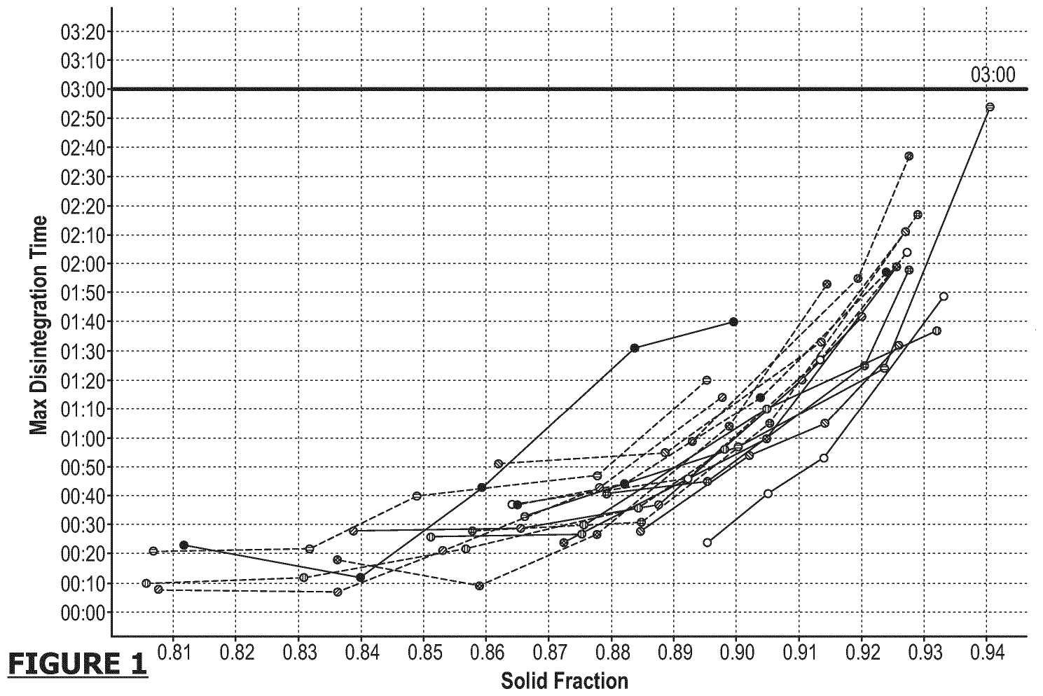

Table 6 and FIG. 1 show that tablets with a solid fraction of no more than 0.9 disperse in less than 3 min. The solid fraction is calculated by dividing the tablet density (g/cm³) by the input material or compression blend true density (g/cm³) and is therefore unitless. The tablet density is calculated by dividing the tablet weight (mg) by the volume of the tablet (mm). The volume of the tablet being determined using the tablet dimensions and geometry. The input compression blend true density can be measured using a Pycnometer or calculated from all individual components true density previously measured or available from the literature.

TABLE 6

| | | Tablets Disintegration Time |
|---|---|---|
| Formulation | Tablets Solid Fraction | (seconds) |
| 2 | 0.81 | 24 |
| | 0.86 | 30 |
| | 0.90 | 48 |
| | 0.93 | 148 |
| | 0.94 | 197 |

The composition of formulation 2 is provided in Example 1.

The lines in FIG. 1 relate to different manufacturing batches of tablets having Formulations 24 or 25 where the tablets in each line differ only in solid fraction.

As per USP<1062>, there is a direct relationship between the tablet tensile strength (MPa) and the tablet solid fraction. Tensile strength (Ts) is expressed in MPa and is calculated from the tablet hardness, thickness and tooling dimensions as shown in the following equation (Pitt and Heasley, 2013, Powder Technology, 238:169-175):

$${T}_{s} = \frac{2}{3} [ \frac{10 P}{π {D}^{2}} {( 2.84 \frac{T}{D} - 0.126 \frac{T}{W} + 3.15 \frac{W}{D} + 0 \text{.01} )}^{- 1} ]$$

where P is the tablet hardness, D is the length of the short axis, T is the tablet thickness and W is the central cylinder thickness (tablet wall height).

In view of the relationship between tablet solid fraction and tablet tensile tablet hardness, and tablet solid fraction and tablet disintegration time, all the formulations disclosed in Examples 1, 4 to 12 and 14 to 16 have a similar tensile strength, and a tablet solid fraction of no more than 0.9.

### Example 3

Table 7 shows that tablets comprising a minimum of 40% w/w granule have acceptable content uniformity with an AV<15 as per USP <905>. Granule loadings ranging from 40 to 75% were found suitable to produce fast dispersing tablets. Granule loading could be further increase successfully up to 83% in the very small size 100 mg core weight dispersible tablet.

TABLE 7

| | Granule loading in tablet | Tablet Content Uniformity |
|---|---|---|
| Formulation | (% w/w) | Acceptance Value |
| 5 | 10 | 21 |
| 6 | 20 | 20 |
| 7 | 40 | 12 |
| 8 | 50 | 6 |
| 9 | 80 | 7 |
| 10 | 83.3 | 5 |

The composition of formulations 5-10 are given in Table 8 and are intended solely as a illustration of the effect of granule content upon content uniformity as they are not dispersible tablets of the invention.

TABLE 8

| | Formula- | Formula- | Formula- | Formula- | Formula- | Formula- |
|---|---|---|---|---|---|---|
| | tion 5 | tion 6 | tion 7 | tion 8 | tion 9 | tion 10 |
| Component | (% w/w) | (% w/w) | (% w/w) | (% w/w) | (% w/w) | (%w/w) |
| Intra-granular | | | | | | |
| Drug | 1.0 | 0.5 | 0.5 | 0.5 | 0.1 | 0.2 |
| substance | | | | | | |
| Mannitol | 72.5 | 73.0 | 73.0 | 73.0 | 73.4 | 73.3 |
| MCC | 20.0 | 20.0 | 20.0 | 20.0 | 20.0 | 20.0 |
| Hypromellose | 5.0 | 5.0 | 5.0 | 5.0 | 5.0 | 5.0 |
| Croscarmellose | 1.5 | 1.5 | 1.5 | 1.5 | 1.5 | 1.5 |
| sodium | | | | | | |
| Total | 100.0 | 100.0 | 100.0 | 100.0 | 100.0 | 100.0 |
| Granule | 10.0 | 20.0 | 40.0 | 50.0 | 80.0 | 83.3 |
| Extra-granular | | | | | | |
| SMCC/MCC | 26.0 | 21.0 | 29.0 | 6.0 | 0 | 0 |
| Mannitol | 44.0 | 39.0 | 11.0 | 24.0 | 0 | 0 |
| L-HPC | 18.0 | 18.0 | 18.0 | 18.0 | 18.0 | 14.7 |
| Sodium | 2.0 | 2.0 | 2.0 | 2.0 | 2.0 | 2.0 |
| stearyl | | | | | | |
| fumarate | | | | | | |
| Total | 100.0 | 100.0 | 100.0 | 100.0 | 100.0 | 100.0 |

### Example 4

Table 9 shows tablets comprising granules with a median size of ≤140 microns disintegrate in less than 3 min at a target mean tablet tensile strength of 2 MPa (+0.5 MPa).

TABLE 9

| | Granule Median Size | Tablets Disintegration Time |
|---|---|---|
| Formulation | (microns) | (seconds) |
| 1 | 90 | 51 |
| 1 | 101 | 44 |
| 1 | 111 | 24 |
| 11 | 140 | 65 |

The composition of formulations 1 and 11 are given in Table 10.

TABLE 10

| | Formulation 1 | Formulation 11 |
|---|---|---|
| Component | (% w/w) | (% w/w) |
| Intra-granular | | |
| Drug substance | 0.3 | 2.0 |
| Mannitol | 74.7 | 35.0 |
| MCC | 20.0 | 55.0 |
| Hypromellose | 2.0 | 0 |
| Povidone | 0 | 5.0 |
| Croscarmellose sodium | 3.0 | 3.0 |
| Total | 100.0 | 100.0 |
| Granule | 75.0 | 75.8 |
| Extra-granular | | |
| SMCC/MCC | 20.5 | 20.2 |
| Croscarmellose sodium | 3.0 | 3.0 |
| Sucralose | 1.0 | 0 |
| Magnesium Stearate | 0.5 | 1.0 |
| Sodium stearyl fumarate | 0 | 0 |
| Total | 100.0 | 100.0 |

### Example 5

Table 11 shows that tablets comprising granule containing 20-60% w/w microcrystalline cellulose (MOC) disperse in less than 3 min at a target mean tablet tensile strength of 2 MPa (+0.5 MPa). The fastest disintegration time was observed with 20% w/w microcrystalline cellulose and this was also optimal for granulation process robustness.

TABLE 11

| | Quantity of intra-granular | Tablets |
|---|---|---|
| | Microcrystalline Cellulose | Disintegration Time |
| Formulation | (% w/w) | (seconds) |
| 1 | 20 | 21 |
| 2 | 20 | 7 |
| 3 | 30 | 30 |
| 3 | 30 | 21 |
| 13 | 60 | 107 |
| 14 | 60 | 63 |

Table 12 gives the compositions of formulations 1, 2, 3, 13, 14.

TABLE 12

| | Formu- | Formu- | Formu- | Formu- | Formu- |
|---|---|---|---|---|---|
| | lation | lation | lation | lation | lation |
| | 1 (% | 2 (% | 3 (% | 13 (% | 14 (% |
| Component | w/w) | w/w) | w/w) | w/w) | w/w) |
| Intra-granular | | | | | |
| Drug substance | 0.3 | 2.7 | 7.9 | 0.3 | 3.3 |
| Mannitol | 74.7 | 72.3 | 55.1 | 32.7 | 29.7 |
| MCC | 20.0 | 20.0 | 30.0 | 60.0 | 60.0 |
| Hypromellose | 2.0 | 2.0 | 3.0 | 4.0 | 4.0 |
| Croscarmellose | 3.0 | 3.0 | 0 | 3.0 | 3.0 |
| sodium | | | | | |
| Sodium starch | 0 | 0 | 4.0 | 0 | 0 |
| glycolate | | | | | |
| Total | 100.0 | 100.0 | 100.0 | 100.0 | 100.0 |
| Granule | 75.0 | 75.0 | 44.4 | 75.1 | 75.1 |
| Extra-granular | | | | | |
| SMCC/MCC | 20.5 | 20.5 | 42.6 | 20.9 | 21.4 |
| Croscarmellose | 3.0 | 3.0 | 0 | 3.0 | 3.0 |
| sodium | | | | | |
| Crospovidone | 0 | 0 | 10.0 | 0 | 0 |
| XL-10 | | | | | |
| Sucralose | 1.0 | 1.0 | 1.0 | 0 | 0 |
| Magnesium | 0.5 | 0.5 | 0 | 1.0 | 0.5 |
| Stearate | | | | | |
| Sodium stearyl | 0 | 0 | 2.0 | 0 | 0 |
| fumarate | | | | | |
| Total | 100.0 | 100.0 | 100.0 | 100.0 | 100.0 |

### Example 6

Table 13 shows that the MCC to binder ratio needs to be ≥10:1 in the granule for the tablets to disintegrate in less than 3 min for tablets comprising high level of granules (75-83% w/w) at a target mean tablet tensile strength of 2 MPa (+0.5 MPa) . . . . The MCC to binder ratio can be ≥4:1 for the tablets to disintegrate in less than 3 min for tablets comprising lower levels of granules (e.g. 40% w/w granules) at a target mean tablet tensile strength of 2 MPa (+0.5 MPa).

TABLE 13

| | MCC:Binder | Granule loading in | Tablets Disintegration |
|---|---|---|---|
| Formulation | Ratio | tablet (% w/w) | Time (seconds) |
| 7 | 4:1 | 40 | 87 |
| 23 | 6:1 | 44 | 43 |
| 22 | 8:1 | 44 | 56 |
| 1 | 10:1 | 75 | 21 |
| 2 | 10:1 | 75 | 7 |
| 13 | 15:1 | 75 | 107 |
| 12 | 20:1 | 75 | 48 |

The composition of formulations 1, 2, 7, 12 and 13 are given in Table 14.

TABLE 14

| | Formula- | Formula- | Formula- | Formula- | Formula- | Formula- | Formula- |
|---|---|---|---|---|---|---|---|
| | tion 1 | tion 2 | tion 12 | tion 13 | tion 7 | tion 22 | tion 23 |
| Component | (% w/w) | (% w/w) | (% w/w) | (% w/w) | (% w/w) | (% w/w) | (% w/w) |
| Intra-granular | | | | | | | |
| Drug | 0.3 | 2.7 | 3.3 | 0.3 | 0.5 | 7.9 | 7.9 |
| substance | | | | | | | |
| Mannitol | 74.7 | 72.3 | 51.7 | 32.7 | 73.0 | 32.1 | 54.1 |
| MCC | 20.0 | 20.0 | 40.0 | 60.0 | 20.0 | 40.0 | 30.0 |
| Hypromellose | 2.0 | 2.0 | 2.0 | 4.0 | 5.0 | 5.0 | 5.0 |
| Croscarmellose | 3.0 | 3.0 | 3.0 | 3.0 | 1.5 | 0 | 0 |
| sodium | | | | | | | |
| Crospovidone | 0 | 0 | 0 | 0 | 0 | 15.0 | 0 |
| XL-10 | | | | | | | |
| Sodium | 0 | 0 | 0 | 0 | 0 | 0 | 3.0 |
| starch | | | | | | | |
| glycolate | | | | | | | |
| Total | 100.0 | 100.0 | 100.0 | 100.0 | 100.0 | 100.0 | 100.0 |
| Granule | 75.0 | 75.0 | 75.1 | 75.1 | 40.0 | 44.4 | 44.4 |
| Extra-granular | | | | | | | |
| SMCC/MCC | 20.5 | 20.5 | 21.4 | 20.9 | 29.0 | 37.6 | 42.6 |
| Mannitol | 0 | 0 | 0 | 0 | 11.0 | 0 | 0 |
| Croscarmellose | 3.0 | 3.0 | 3.0 | 3.0 | 3.0 | 0 | 0 |
| sodium | | | | | | | |
| L-HPC | 0 | 0 | 0 | 0 | 18.0 | 0 | 0 |
| Crospovidone | 0 | 0 | 0 | 0 | 0 | 15.0 | 10.0 |
| XL-10 | | | | | | | |
| Sucralose | 1.0 | 1.0 | 0 | 0 | 0 | 1.0 | 1.0 |
| Magnesium | 0.5 | 0.5 | 0.5 | 1.0 | 0 | 0 | 0 |
| Stearate | | | | | | | |
| Sodium | 0 | 0 | 0 | 0 | 2.0 | 2.0 | 2.0 |
| stearyl | | | | | | | |
| fumarate | | | | | | | |
| Total | 100.0 | 100.0 | 100.0 | 100.0 | 100.0 | 100.0 | 100.0 |

### Example 7

Table 15 shows that tablets comprising ≥20% silicified microcrystalline cellulose (SMCC) disperse in less than 3 min at a target mean tablet tensile strength of 2 MPa (+0.5 MPa).

TABLE 15

| | Quantity of extra- | Tablets Disintegration |
|---|---|---|
| Formulation | granular SMCC (% w/w) | Time (seconds) |
| 16 | 11.7 | 290 |
| 11 | 20.2 | 65 |
| 1 | 20.5 | 21 |
| 2 | 20.5 | 7 |
| 17 | 24.6 | 41 |

Table 16 shows the composition of formulations 1, 2, 11, 16 and 17.

TABLE 16

| | Formu- | Formu- | Formu- | Formu- | Formu- |
|---|---|---|---|---|---|
| | lation | lation | lation | lation | lation |
| | 1 (% | 2 (% | 11 (% | 16 (% | 17 (% |
| Component | w/w) | w/w) | w/w) | w/w) | w/w) |
| Intra-granular | | | | | |
| Drug substance | 0.3 | 2.7 | 2.0 | 2.0 | 2.0 |
| Mannitol | 74.7 | 72.3 | 35.0 | 35.0 | 35.0 |
| MCC | 20.0 | 20.0 | 55.0 | 55.0 | 55.0 |
| Hypromellose | 2.0 | 2.0 | 0 | 0 | 0 |
| Povidone | 0 | 0 | 5.0 | 5.0 | 5.0 |
| Croscarmellose | 3.0 | 3.0 | 3.0 | 3.0 | 3.0 |
| sodium | | | | | |
| Total | 100.0 | 100.0 | 100.0 | 100.0 | 100.0 |
| Granule | 75.0 | 75.0 | 75.8 | 83.3 | 71.4 |
| Extra-granular | | | | | |
| SMCC/MCC | 20.5 | 20.5 | 20.2 | 11.7 | 24.6 |
| Croscarmellose | 3.0 | 3.0 | 3.0 | 3.0 | 3.0 |
| sodium | | | | | |
| Sucralose | 1.0 | 1.0 | 1.0 | 0 | 0 |
| Magnesium | 0.5 | 0.5 | 0 | 0 | 1.0 |
| Stearate | | | | | |
| Sodium stearyl | 0 | 0 | 0 | 2.0 | 0 |
| fumarate | | | | | |
| Total | 100.0 | 100.0 | 100.0 | 100.0 | 100.0 |

### Example 8

Table 17 shows that tablets comprising croscarmellose sodium, crospovidone XL-10, sodium starch glycolate and L-HPC as extra-granular disintegrants disperse in less than 3 min at a target mean tablet tensile strength of 2 MPa (+0.5 MPa).

Crospovidone XL-10 disintegrant may be used at a level of 10-15% w/w in the extra-granular composition of the dispersible tablet.

TABLE 17

| | Type of | Quantity of | Tablets |
|---|---|---|---|
| | extra-granular | extra-granular | Disintegration |
| Formulation | disintegrant | disintegrant | Time (seconds) |
| 1 | Croscarmellose sodium | 3.0 | 21 |
| 2 | Croscarmellose sodium | 3.0 | 7 |
| 3 | Crospovidone XL-10 | 10.0 | 30 |
| 21 | Crospovidone XL-10 | 15.0 | 57 |
| 15 | Sodium starch glycolate | 5.0 | 20 |
| 26 | L-HPC | 15.0 | 25 |

The composition of formulations 1, 2, 3, 21, 26 and 15 are shown in Table 18. It is noted that even though formulations 15, 21 and 26 are not tablets of the invention, they nonetheless rapidly disperse and provide support for the use of sodium starch glycolate and L-HPC as extragranular disintegrants.

TABLE 18

| | Formula- | Formula- | Formula- | Formula- | Formula- | Formula- |
|---|---|---|---|---|---|---|
| | tion 1 | tion 2 | tion 3 | tion 15 | tion 21 | tion 26 |
| Component | (% w/w) | (% w/w) | (% w/w) | (% w/w) | (% w/w) | (% w/w) |
| Intra-granular | | | | | | |
| Drug | 0.3 | 2.7 | 7.9 | 7.9 | 7.9 | 7.9 |
| substance | | | | | | |
| Mannitol | 74.7 | 72.3 | 55.1 | 54.1 | 54.1 | 37.1 |
| MCC | 20.0 | 20.0 | 30.0 | 30.0 | 30.0 | 40.0 |
| Hypromellose | 2.0 | 2.0 | 3.0 | 4.0 | 4.0 | 5.0 |
| Croscarmellose | 3.0 | 3.0 | 0 | 0 | 0 | 0 |
| sodium | | | | | | |
| Sodium starch | 0 | 0 | 4.0 | 4.0 | 4.0 | 0 |
| glycolate | | | | | | |
| Crospovidone | 0 | 0 | 0 | 0 | 0 | 10.0 |
| XL-10 | | | | | | |
| Total | 100.0 | 100.0 | 100.0 | 100.0 | 100.0 | 100.0 |
| Granule | 75.0 | 75.0 | 44.4 | 44.4 | 44.4 | 44.4 |
| Extra-granular | | | | | | |
| SMCC/MCC | 20.5 | 20.5 | 42.6 | 47.6 | 37.6 | 37.6 |
| Croscarmellose | 3.0 | 3.0 | 0 | 0 | 0 | 0 |
| sodium | | | | | | |
| Crospovidone | 0 | 0 | 10.0 | 0 | 15.0 | 0 |
| XL-10 | | | | | | |
| Sodium starch | 0 | 0 | 0 | 5.0 | 0 | 0 |
| glycolate | | | | | | |
| L-HPC | 0 | 0 | 0 | 0 | 0 | 15.0 |
| Sucralose | 1.0 | 1.0 | 1.0 | 1.0 | 1.0 | 1.0 |
| Magnesium | 0.5 | 0.5 | 0 | 0 | 0 | 0 |
| Stearate | | | | | | |
| Sodium | 0 | 0 | 2.0 | 2.0 | 2.0 | 2.0 |
| stearyl | | | | | | |
| fumarate | | | | | | |
| Total | 100.0 | 100.0 | 100.0 | 100.0 | 100.0 | 100.0 |

### Example 9

Table 19 shows that tablets comprising 3 to 6% w/w extra-granular croscarmellose sodium disintegrant disperse in less than 3 min at a target mean tablet tensile strength of 2 MPa (+0.5 MPa).

TABLE 19

| | Quantity of extra-granular | Tablets |
|---|---|---|
| | croscarmellose sodium | Disintegration Time |
| Formulation | disintegrant (% w/w) | (seconds) |
| 18 | 3 | 102 |
| 1 | 3 | 21 |
| 2 | 3 | 7 |
| 19 | 6 | 109 |

The composition of formulations 1, 2, 18 and 19 are shown in Table 20.

TABLE 20

| | Formu- | Formu- | Formu- | Formu- |
|---|---|---|---|---|
| | lation 1 | lation 2 | lation 18 | lation 19 |
| Component | (% w/w) | (% w/w) | (% w/w) | (% w/w) |
| Intra-granular | | | | |
| Drug substance | 0.3 | 2.7 | 0.3 | 0.3 |
| Mannitol | 74.7 | 72.3 | 32.7 | 32.7 |
| MCC | 20.0 | 20.0 | 60.0 | 60.0 |
| Hypromellose | 2.0 | 2.0 | 4.0 | 4.0 |
| Croscarmellose | 3.0 | 3.0 | 3.0 | 3.0 |
| sodium | | | | |
| Total | 100.0 | 100.0 | 100.0 | 100.0 |
| Granule | 75.0 | 75.0 | 70.0 | 70.0 |
| Extra-granular | | | | |
| SMCC/MCC | 20.5 | 20.5 | 26.0 | 23.0 |
| Croscarmellose | 3.0 | 3.0 | 3.0 | 6.0 |
| sodium | | | | |
| Sucralose | 1.0 | 1.0 | 0 | 0 |
| Magnesium | 0.5 | 0.5 | 1.0 | 1.0 |
| Stearate | | | | |
| Total | 100.0 | 100.0 | 100.0 | 100.0 |

### Example 10

Table 21 shows that tablets comprising 10 to 15% w/w extra-granular crospovidone XL-10 disintegrant disperse in less than 3 min at a target mean tablet tensile strength of 2 MPa (+0.5 MPa).

TABLE 21

| | Quantity of extra-granular | Tablets |
|---|---|---|
| | crospovidone XL-10 | Disintegration Time |
| Formulation | disintegrant (% w/w) | (seconds) |
| 20 | 10 | 105 |
| 3 | 10 | 21 |
| 21 | 15 | 57 |

Table 22 provides the compositions of formulations 3, 20 and 21. It is noted that even though formulations 20 and 21 are not tablets of the invention, they nonetheless rapidly disperse and provide support for the use of crospovidone XL-10 extragranularly in an amount of 10%.

TABLE 22

| | Formu- | Formu- | Formu- |
|---|---|---|---|
| | lation 3 | lation 20 | lation 21 |
| Component | (% w/w) | (% w/w) | (% w/w) |
| Intra-granular | | | |
| Drug substance | 7.9 | 0.1 | 7.9 |
| Mannitol | 55.1 | 31.9 | 54.1 |
| MCC | 30.0 | 60.0 | 30.0 |
| Hypromellose | 3.0 | 0 | 4.0 |
| Povidone | 0 | 3.0 | 0 |
| Sodium starch glycolate | 4.0 | 0 | 4.0 |
| Crospovidone XL-10 | 0 | 5.0 | 0 |
| Total | 100.0 | 100.0 | 100.0 |
| Granule | 44.4 | 83.3 | 44.4 |
| Extra-granular | | | |
| SMCC/MCC | 42.6 | 4.7 | 37.6 |
| Crospovidone XL-10 | 10.0 | 10.0 | 15.0 |
| Sucralose | 1.0 | 0 | 1.0 |
| Sodium stearyl | 2.0 | 2.0 | 2.0 |
| fumarate | | | |
| Total | 100.0 | 100.0 | 100.0 |

### Example 11

Table 23 shows that tablets comprising a second intra-granular soluble filler in addition to MCC have lower amounts of insoluble solids and therefore an improved mouthfeel. Granules comprising 17 to 75% w/w soluble filler (e.g mannitol) have greater mechanical strength (lower level of fines) for downstream processing into tablets.

Disintegration is measured at a target mean tablet tensile strength of 2 MPa (+0.5 MPa).

TABLE 23

| | | | | % fines in | |
|---|---|---|---|---|---|
| | | | | the | |
| | | | Quantity | granules - | |
| | | | of intra- | Surrogate | |
| | Quantity | Quantity | granular | for | |
| | of | of | insoluble | granule | |
| | soluble | insoluble | solids/MCC | mechanical | |
| | intra- | intra- | (mg/unit | strength, | Tablets |
| | granular | granular | dose) - | manufac- | Disinte- |
| | filler | filler | Surrogate | turing | gration |
| Formu- | (mannitol) | (MCC) | for | process | Time |
| lation | (% w/w) | (% w/w) | mouthfeel | robustness | (seconds) |
| 1 | 75 | 20 | 15 | 7.9 | 21 |
| 3 | 55 | 30 | 20 | 6.3 | 30 |
| 13 | 33 | 60 | 45 | 7.2 | 107 |
| 4 | 17 | 20 | 145.6 | 2.3 | 116 |

The composition of formulations 1, 3, 4 and 13 are shown in Table 24.

TABLE 24

| | Formu- | Formu- | Formu- | Formu- |
|---|---|---|---|---|
| | lation 1 | lation 3 | lation 4 | lation 13 |
| Component | (% w/w) | (% w/w) | (% w/w) | (% w/w) |
| Intra-granular | | | | |
| Drug substance | 0.3 | 7.9 | 57.8 | 0.3 |
| Mannitol | 74.7 | 55.1 | 17.2 | 32.7 |
| MCC | 20.0 | 30.0 | 20.0 | 60.0 |
| Hypromellose | 2.0 | 3.0 | 2.0 | 4.0 |
| Croscarmellose | 3.0 | 0 | 3.0 | 3.0 |
| sodium | | | | |
| Sodium starch | 0 | 4.0 | 0 | 0 |
| glycolate | | | | |
| Total | 100.0 | 100.0 | 100.0 | 100.0 |
| Granule | 75.0 | 44.4 | 56.0 | 75.1 |
| Extra-granular | | | | |
| SMCC/MCC | 20.5 | 42.6 | 33.0 | 20.9 |
| Croscarmellose | 3.0 | 0 | 6.0 | 3.0 |
| sodium | | | | |
| Crospovidone XL-10 | 0 | 10.0 | 0 | 0 |
| Sucralose | 1.0 | 1.0 | 1.5 | 0 |
| Acesulfame | 0 | 0 | 3.0 | 0 |
| potassium | | | | |
| Magnesium | 0.5 | 0 | 0 | 1.0 |
| Stearate | | | | |
| Sodium stearyl | 0 | 2.0 | 0.5 | 0 |
| fumarate | | | | |
| Total | 100.0 | 100.0 | 100.0 | 100.0 |

### Example 12

Table 25 shows that tablets comprising an intra-granular disintegrant (e.g. croscarmellose sodium, sodium starch glycolate and crospovidone XL-10) disperse in less than 3 min at a target mean tablet tensile strength of 2 MPa (+0.5 MPa) . . . 3% w/w croscarmellose sodium and 4% sodium starch glycolate were successfully used in the granules to produce fast dispersing drug products (dispersing in 30 s).

TABLE 25

| | | Quantity of | Tablets |
|---|---|---|---|
| | Intra- | intra-granular | Disinte- |
| | granular | disintegrant | gration |
| Formulation | disintegrant | (% w/w) | Time (seconds) |
| 1 | Croscarmellose | 3.0 | 21 |
| | sodium | | |
| 3 | Sodium starch | 4.0 | 30 |
| | glycolate | | |
| 20 | Crospovidone XL-10 | 5.0 | 105 |

Table 26 shows the compositions of formulations 1, 3 and 20. Whilst the SMCC content of formulation 20 is <20%, it is noted that this tablet nonetheless meets the disintegration time test, and can demonstrate that a variety of disintegrants can be used intragranularly.

TABLE 26

| | Formulation 1 | Formulation 3 | Formulation 20 |
|---|---|---|---|
| Component | (% w/w) | (% w/w) | (% w/w) |
| Intra-granular | | | |
| Drug substance | 0.3 | 7.9 | 0.1 |
| Mannitol | 74.7 | 55.1 | 31.9 |
| MCC | 20.0 | 30.0 | 60.0 |
| Hypromellose | 2.0 | 3.0 | 0 |
| Povidone | | | |
| Croscarmellose | 3.0 | 0 | 0 |
| sodium | | | |
| Sodium starch | 0 | 4.0 | 0 |
| glycolate | | | |
| Crospovidone XL-10 | 0 | 0 | 5.0 |
| Total | 100.0 | 100.0 | 100.0 |
| Granule | 75.0 | 44.4 | 83.3 |
| Extra-granular | | | |
| SMCC/MCC | 20.5 | 42.6 | 4.7 |
| Croscarmellose | 3.0 | 0 | 0 |
| sodium | | | |
| Crospovidone XL-10 | 0 | 10.0 | 10.0 |
| Sucralose | 1.0 | 1.0 | 0 |
| Magnesium | 0.5 | 0 | 0 |
| Stearate | | | |
| Sodium stearyl | 0 | 2.0 | 2.0 |
| fumarate | | | |
| Total | 100.0 | 100.0 | 100.0 |

### Example 13

Table 27 shows that tablets comprising no more than 200 mg of MCC and SMCC have an acceptable mouthfeel. The MCC and SMCC grades used had a small size of no more than 125 microns for improved mouthfeel. The mouthfeel is considered acceptable when the statistical mean response from the taste study participants is ‘acceptable’ and with the majority of the participants finding the mouthfeel ‘acceptable’.

TABLE 27

| | | | | | Mouthfeel |
|---|---|---|---|---|---|
| | | | % of | Volume | (from Human |
| Paediatric | Tablet | | combined | for | Sensory |
| Dispersible | size | % | MCC and | dispersion | Analysis |
| Tablet | (mg) | API | SMCC | (mL) | studies) |
| 27 | 90 | 5.8 | 60 | 5 | Acceptable |
| | | | 54 mg/tab | | |
| 28 | 100 | 2 | 31 | 5 | Acceptable |
| | | | 31 mg/tab | | |
| 29 | 150 | 23.5 | 48.9 | 15 | Acceptable |
| | | | 72 mg/tab | | |
| 30 | 150 | 3.5 | 55.9 | 5 | Acceptable |
| | | | 83.8 mg/tab | | |
| 31 | 250 | 40 | 36 | 5 | Acceptable |
| | | | 90.55 mg/tab | | |
| 32 | 314 | 20 | 63 | 5 | Acceptable |
| | | | 197 mg/tab | | |
| 33 | 380 | 28 | 53 | 15-20 | Acceptable |
| | | | 200 mg/tab | | |
| 34 | 1300 | 32 | 44 | 10 | Not |
| | | | 575 mg/tab | | acceptable |

### Example 14

Table 28 show that multiple tablets of the invention can disperse in 5 mL water. The API in formulations 24 and 25 is daprodustat free acid. Formulation 24 is a 0.25 mg daprodustat dispersible tablet and formulation 25 is a 2 mg daprodustat dispersible tablet.

TABLE 28

| | Number of | Volume | Tablets |
|---|---|---|---|
| | tablets | of Water | Disintegration |
| Formulation | dispersed | (mL) | Time (seconds) |
| 24 | 3 | 5 | 60 |
| | 4 | 5 | 59 |
| | 6 | 10 | 65 |
| 25 | 3 | 5 | 62 |
| | 4 | 5 | 57 |
| | 6 | 10 | 55 |
| | 8 | 10 | 62 |

The composition of formulations 24 and 25 is given in table 29. Note formulation 24 is a film coated version of formulation 1 and formulation 25 is a film coated version of formulation 2. Note that the purified water is removed during processing. Note also that the weight of film coat applied per tablet may vary depending upon the efficiency of the process but is typically 3.0% w/w of the tablet core weight.

TABLE 29

Formulation

| | |
|---|---|
| 24 | 25 |

Component
Quantity
(mg/tablet)

| | | |
|---|---|---|
| Strength (mg) | 0.25 | 2 |

Intragranular Components

| | | | |
|---|---|---|---|
| Active Substance | Daprodustat | 0.25 | 2.00 |
| | free acid | | |
| Filler | Mannitol | 56.00 | 54.25 |
| Insoluble | Microcrystalline | 15.00 | 15.00 |
| excipient | Cellulose | | |
| Disintegrant | Croscarmellose | 2.25 | 2.25 |
| | Sodium | | |
| Binder | Hypromellose | 1.50 | 1.50 |
| Granulation | Purified | — | — |
| fluid | Water | | |

Extragranular Components

| | | | |
|---|---|---|---|
| Insoluble | Silicified | 20.50 | 20.50 |
| excipient | Microcrystalline | | |
| | Cellulose | | |
| Disintegrant | Croscarmellose | 3.00 | 3.00 |
| | Sodium | | |
| Sweetener | Sucralose | 1.00 | 1.00 |
| Lubricant | Magnesium | 0.50 | 0.50 |
| | Stearate | | |

Film-coat Components

| | | | |
|---|---|---|---|
| Film coat | Opadry ® Green | 3.00 | — |
| | 05B210001 | | |
| Film coat | Opadry ® White | — | 3.00 |
| | 05B280000 | | |
| Film coat | Purified | — | — |
| vehicle | Water² | | |

| | | |
|---|---|---|
| Total unit dose | 103.00 | 103.00 |

### Example 15

60 Tablets (formulation 24 in Table 30 and formulation 25 in Table 31) were packed in a 60 cc HDPE bottle with induction seal cap and 2 g desiccant. Stability of the tablets was assessed under a range of conditions (RH=relative humidity; Amb=ambient; Exposed=tablets outside of primary container e.g. open in a petri dish).

TABLE 30

| | | | Total | |
|---|---|---|---|---|
| | | Daprodustat | daprodustat- | |
| | | content | related | Water |
| Storage | Time | (% label | impurities | Content |
| Condition | (Months) | claim) | (% area) | (% w/w) |
| Initial | 0 | 100.1 | <0.06 | 1.0 |
| 30° C./75% RH | 3 | 99.6 | <0.06 | 1.0 |
| 40° C./75% RH | 3 | 99.7 | <0.06 | 1.2 |
| 50° C./Amb | 28 days | 99.7 | <0.06 | 1.2 |
| | 3 | 98.8 | 0.18 | 0.9 |
| Freeze-Thaw | 28 days | 99.4 | <0.06 | 0.9 |
| 30° C./75% RH | 28 days | 99.2 | <0.06 | 4.4 |
| Exposed | 60 days | 99.4 | 0.06 | 4.3 |

TABLE 31

| | | | Total | |
|---|---|---|---|---|
| | | | daprodustat- | |
| | | Daprodustat | related | Water |
| Storage | Time | content | impurities | Content |
| Condition | (Months) | (%) | (% area) | (% w/w)⁴ |
| Initial | 0 | 97.0 | <0.06 | 1.0 |
| 30° C./75% RH | 3 | 96.7 | <0.06 | 1.0 |
| 40° C./75% RH | 3 | 97.6 | <0.06 | 1.1 |
| 50° C./Amb | 28 days | 96.4 | <0.06 | 1.1 |
| | 3 | 97.0 | 0.08 | 0.9 |
| Freeze-Thaw | 28 days | 96.7 | <0.06 | 0.9 |
| 30° C./75% RH | 28 days | 96.5 | <0.06 | 4.3 |
| Exposed | 60 days | 96.2 | <0.06 | 4.3 |

Tables 30 and 31 demonstrate that formulations 24 and 25 have good stability when stored under conditions of 40° C./75% RH for 3 months.

### Example 16

Dispersions (1×0.25 mg tablet (formulation 24) and 4×2 mg tablets (formulation 25) were prepared by adding water, then tablets, then swirling for 2 minutes in the following dosing devices:
30 mL dosing cup
   Glass (soda lime)

$$10 mL syringe + cup$$

$$Gastronasal ( GN ) tube + syringe + cup$$

Dose recovery was assessed by measuring drug content for each dispersion. The drug content of a 5 mL rinse was also measured. Acceptable recovery is considered to be not less than 90% LC for the total combined content of the recovery sample and rinse sample. Table 32 shows that acceptable recovery was demonstrated for all doses, dosing devices and dosing device combinations tested. Syringes and GN feeding tubes show less than 90% recovery from the recovery sample, suggesting a rinse step is important to ensure recovery >90% is achieved where these dosing devices are used.

TABLE 32

| | | Mean | Mean | Mean |
|---|---|---|---|---|
| | | Recovery | Rinse | Combined |
| | | Content | Content | Content |
| Strength | Dosing | (% LC, | (% LC, | (% LC, |
| (mg) | Device(s) | n = 3) | n = 3) | n = 3) |
| 0.25 | Cup | 94.7 | 3.1 | 97.8 |
| | Glass | 93.9 | 5.6 | 99.5 |
| | Syringe + Cup | 83.7 | 9.5 | 93.2 |
| | PVC GN Feeding | 77.5 | 13.5 | 91.0 |
| | Tube + Syringe + | | | |
| | Cup | | | |
| | Polyurethane GN | 84.5 | 9.5 | 94.0 |
| | Feeding Tube + | | | |
| | Syringe + Cup | | | |
| 2 | Cup | 99.0 | 3.2 | 102.2 |
| | Glass | 96.7 | 4.8 | 101.4 |
| | Syringe + Cup | 94.6 | 5.3 | 99.9 |
| | PVC GN Feeding | 91.2 | 8.8 | 100.0 |
| | Tube + Syringe + | | | |
| | Cup | | | |
| | Polyurethane GN | 92.9 | 7.3 | 100.2 |
| | Feeding Tube + | | | |
| | Syringe + Cup | | | |

### Example 17

A dispersion (1×0.25 mg tablet (formulation 24)) was prepared by adding water then swirling for 2 minutes in the following dosing devices:
30 mL dosing cup
Glass (soda lime)

Stability of the drug substance in the dispersion was assessed by measuring content and impurities at the following time points: 0, 30 minutes, 60 minutes and 120 minutes. Table 33 shows that the dispersion was stable up to 120 minutes.

TABLE 33

| Strength | Dosing | Content (% LC, n = 3) | | | | Impurities (Greatest Impurity from n = 3) | | | |
|---|---|---|---|---|---|---|---|---|---|
| (mg) | Device(s) | INT | 30 min | 60 min | 120 min | INT | 30 min | 60 min | 120 min |
| 0.25 | Cup | 93.9 | 90.1 | 93.0 | 93.4 | ND | ND | 0.09% | ND |
| | | | | | | | | a/a | |
| | Glass | 94.0 | 95.5 | 94.6 | 91.4 | ND | ND | ND | 0.07% |
| | | | | | | | | | a/a |

### Example 18

Table 34 shows that tablets comprising granules with a density of 0.4 to 0.6 mg/ml disintegrate in less than 3 minutes. In particular, tablets comprising granules with a density of 0.5 to 0.6 mg/ml disintegrate in less than 60 sec.

TABLE 34

| | | Tablets |
|---|---|---|
| | Granule | Disintegration |
| Formulation | density | Time (seconds) |
| 1 | 0.52 | 51 |
| | 0.55 | 44 |
| | 0.60 | 24 |

The composition of formulation 1 is given in Table 5.

1. A dispersible tablet exhibiting a solid fraction of less than or equal to 0.9 and a surface area no greater than 615 mm², which dispersible tablet comprises an extra-granular component and at least 40% (w/w in the dispersible tablet) granules, wherein:
a. the granules are ≤140 microns in diameter with a density of 0.4-0.6 g/mL and comprise up to 60% (w/w in the granules) active pharmaceutical ingredient, between 20-60% (w/w in the granules) microcrystalline cellulose and a binder, wherein the ratio of microcrystalline cellulose to the binder is ≥10:1; and
b. the extragranular component comprises at least 20% (w/w in the dispersible tablet) silicified microcrystalline cellulose and a disintegrant.

2. A dispersible tablet according to claim 1, wherein the granules comprise between 20-30% (w/w) microcrystalline cellulose.

3. A dispersible tablet according to claim 2, wherein the granules comprise approximately 20% (w/w) microcrystalline cellulose.

4. A dispersible tablet according to claim 1, wherein the binder is selected from hydroxypropylmethylcellulose or povidone.

5. A dispersible tablet according to claim 1, wherein the disintegrant in the extragranular component is croscarmellose sodium or crospovidone XL-10.

6. A dispersible tablet according to claim 5, wherein croscarmellose sodium is used in an amount of approximately 3-6% w/w in the extra-granular component.

7. A dispersible tablet according to claim 1, wherein the granules additionally comprise one or more soluble fillers that exhibit a solubility of at least 200 mg/ml at 25° C.

8. A dispersible tablet according to claim 7, wherein the granules comprise one soluble filler that is mannitol.

9. A dispersible tablet according to claim 1, wherein the granules additionally comprise a disintegrant.

10. A dispersible tablet according to claim 1, wherein the extragranular component additionally comprises a sweetener and/or a lubricant.

11. A dispersible tablet according to claim 1, wherein one or more tablets capable of complete disintegration in 5 ml water within a time period of 3 minutes.

12. A dispersible tablet according to claim 11, wherein the suspension prepared following complete disintegration contains less than 200 mg insoluble solid particles having a d50 not greater than 125 microns per unit dose dispersed.

13. A dispersible tablet according to claim 1, wherein the active pharmaceutical ingredient is daprodustat or a pharmaceutically acceptable salt thereof.

14. A dispersible tablet exhibiting a solid fraction of less than or equal to 0.9 and a surface area no greater than 615 mm², which dispersible tablet comprises an extra-granular component and 70-83% % (w/w in the dispersible tablet) granules, wherein:
a. the granules are ≤140 microns in diameter with a density of 0.4-0.6 g/mL and consist of:
i. up to 3% (w/w in the granules) daprodustat or a pharmaceutically acceptable salt thereof (measured as the free acid);
ii. between 20-30% (w/w in the granules) microcrystalline cellulose;
iii. hydroxypropylmethylcellulose, wherein the ratio of microcrystalline cellulose to hydroxypropylmethylcellulose is 10:1;
iv. a disintegrant that is croscarmellose sodium or crospovidone XL-10;
v. a soluble filler that is mannitol; and
b. the extragranular component consists of:
i. at least 20% (w/w in the dispersible tablet) silicified microcrystalline cellulose;
ii. a disintegrant selected from the group consisting of croscarmellose sodium and crospovidone XL-10;
iii. a lubricant selected from the group consisting of magnesium stearate and sodium stearyl fumarate; and
iv. a sweetener that is sucralose.
